(19) United States
(12) Patent Application Publication
(10) Pub. No.: US 2026/0248861 A1
(43) Pub. Date: Aug. 27, 2026

(54) SOLID DOSAGE FORMS INCLUDING PORCINE THYROID POWDER AND METHODS OF MAKING AND USING THE SAME

(71) Applicant: GENUS LIFESCIENCES INC., Allentown, PA (US)

(72) Inventors: Michael Libman, Southampton, PA (US); Jeffrey M. Moshal, Allentown, PA (US)

(21) Appl. No.: 19/396,601

(22) Filed: Nov. 21, 2025

Related U.S. Application Data
(63) Continuation of application No. 19/153,499, filed on Feb. 6, 2024.
(60) Provisional application No. 63/483,644, filed on Feb. 7, 2023.
(60) Provisional application No. 63/484,369, filed on Feb. 10, 2023.

(51) Int. Cl. A61K 35/55; A61J 1/03; A61J 1/14; A61K 9/00; A61K 9/20

(52) U.S. Cl. CPC A61K 35/55; A61J 1/03; A61J 1/1418; A61K 9/0053; A61K 9/2009; A61K 9/2013; A61K 9/2054; A61K 9/2059

(57) ABSTRACT

Oral solid dosage forms including porcine thyroid powder and methods of making and using the oral solid dosage forms are disclosed.

### FIELD OF THE TECHNOLOGY

The present disclosure relates to oral solid dosage forms comprising porcine thyroid powder and to methods of making and using the same.

### DESCRIPTION OF THE BACKGROUND OF THE TECHNOLOGY

Hypothyroidism is a common endocrine disorder characterized by a deficiency of thyroid hormone. Worldwide, iodine deficiency is the major cause of hypothyroidism. In the United States and other areas in which iodine intake is adequate, autoimmune thyroid disease (Hashimoto's disease) is the most common cause of hypothyroidism. Hypothyroidism may also be drug-induced or otherwise iatrogenic.

Hypothyroidism usually is a primary process in which the thyroid gland is unable to produce sufficient amounts of thyroid hormone. Hypothyroidism can also be a secondary process in which the thyroid gland is normal but receives insufficient stimulation because of low secretion of thyrotropin (i.e., thyroid-stimulating hormone or “TSH”) by the pituitary gland. Also, in tertiary hypothyroidism, the hypothalamus releases inadequate levels of thyrotropin-releasing hormone (TRH), which leads to insufficient release of TSH, and in turn causes inadequate thyroid stimulation.

Thyroid hormone deficiency has a wide range of effects. Systemic effects result from either derangements in metabolic processes or directly by myxedematous infiltration (i.e., accumulation of glucosaminoglycans in the tissues). Hypothyroid changes in the heart result in decreased contractility, cardiac enlargement, pericardial effusion, decreased pulse, and decreased cardiac output. In the gastrointestinal tract, achlorhydria and prolonged intestinal transit time with gastric stasis can occur. Delayed puberty, anovulation, menstrual irregularities, and infertility are common. Decreased thyroid hormone also can cause increased levels of total cholesterol and low-density lipoprotein (LDL) cholesterol and a possible change in high-density lipoprotein (HDL) cholesterol because of a change in metabolic clearance. In addition, hypothyroidism may result in an increase in insulin resistance.

Third-generation TSH assays are readily available and are generally the most sensitive screening tool for primary hypothyroidism. The generally accepted reference range for normal serum TSH is 0.40-4.2 mIU/L. If TSH levels are above the reference range, the next step is to measure free thyroxine (T4). Subclinical hypothyroidism, also referred to as mild hypothyroidism, is defined as normal serum levels of free T4 and triiodothyronine (T3) with a slightly high serum TSH concentration.

Daily administration of thyroid hormone drugs is the standard treatment for hypothyroidism in humans and other mammals. An oral medication such as levothyroxine or a levothyroxine salt can restore adequate hormone levels, reversing the symptoms of hypothyroidism. In general, hypothyroidism can be adequately treated with a constant daily dose of levothyroxine (LT4). The typical human daily dosage of a levothyroxine drug is low, ranging from 25 to 300 μg. The treatment goals for hypothyroidism are to reverse clinical progression and correct metabolic derangements, as evidenced by normal blood levels of TSH and T4.

Careful dosing of levothyroxine drugs is critical to avoid over and under treatment. Symptoms of overtreatment can include, for example, tachycardia, palpitations, atrial fibrillation, nervousness, tiredness, headache, increased excitability, sleeplessness, tremors, and possible angina. Dosage resulting in under treatment of patients can result in, for example, fatigue, cold intolerance, dry skin, hair loss, muscle pain, joint pain, weakness in the extremities, mental impairment, decreased perspiration, paresthesia and nerve entrapment syndromes, blurred vision, and hearing impairment.

Thyroid hormone replacement is commonly started at anticipated full replacement dosage level in young and otherwise healthy patients. In elderly patients and those with known ischemic heart disease, treatment often begins with one-quarter to one-half anticipated full replacement dosage, and dosage is adjusted upwardly in small increments after no less than 4 to 6 weeks. For most cases of mild to moderate hypothyroidism, a starting levothyroxine dosage is 50-75 μg/day.

All thyroid hormone preparations, natural or synthetic, are intended to replace a patient's natural thyroid hormone. A normal human thyroid gland releases primarily tetraiodothyronine (levothyroxine or LT4), which is then converted to triiodothyronine (liothyronine or T3) in the target tissues. T3 is the actual active thyroid hormone. The thyroid gland forms LT4 (containing four iodine atoms) by coupling two molecules of diiodotyrosine (DIT). T3 (containing three iodine atoms) is also formed in the thyroid gland by coupling one molecule of DIT with one molecule of monoiodotyrosine (MIT). The thyroid gland stores both T4 and T3 in the thyroid colloid as thyroglobulin.

The normal thyroid gland contains approximately 200 μg of levothyroxine (T4) per gram of gland and 15 μg of liothyronine (T3) per gram. The ratio of these two hormones in the circulation does not represent the ratio in the thyroid gland since about 80 percent of peripheral liothyronine (T3) comes from monodeiodination of levothyroxine (T4). Peripheral monodeiodination of levothyroxine (T4) at the 5 positions (inner ring) also results in the formation of reverse liothyronine (T3), which is calorigenically inactive.

Synthetic thyroid hormone preparations are commercially available for both T3 and T4 forms of thyroid hormone. For example, liothyronine is a synthetic version of the T3 thyroid hormone triiodothyronine available as CYTOMEL® tablets from King Pharmaceuticals, St. Louis, MO. Levothyroxine (T4) sodium is available in several commercial products including LEVOXYL® tablets (King Pharmaceuticals), UNITHROID® tablets (Jerome Stevens Pharmaceuticals, Bohemia, NY), and SYNTHROID® tablets (AbbVie, Chicago, IL). Levothyroxine (T4) sodium is also available in a veterinary preparation known as SOLOXINE® (King Pharmaceuticals).

Levothyroxine is relatively unstable and degrades over time. For example, levothyroxine sodium is sensitive to irradiation, hydrolysis, oxidation, and heat. Degradation of the active ingredients in thyroxine dosage forms can lead to loss of potency, resulting in inadequate dosage delivery and under treatment of hypothyroidism. Some drug manufacturers have resorted to including an excess of the active ingredient in thyroxine dosage forms in anticipation of some degree of storage degradation. This practice can complicate accurate dosing and present the possibility of over dosing. Accordingly, storage stability of levothyroxine drugs is highly desirable to ensure accurate patient dosing and avoid the complications associated with under or over treatment.

Levothyroxine tablets are available in extremely low, microgram-level dosages (e.g., 25 μg, 50 μg, 75 μg, 88 μg, 100 μg, 112 μg, 125 μg, 137 μg, 150 μg, 175 μg, 200 μg, and 300 μg). To facilitate accurate administration, commercially available tablets typically include a break-line allowing one to break tablets into two substantially equal halves. The possibility that a patient will utilize break-lines and administer only half of a tablet daily enhances the need for uniform distribution of active ingredients in tablets to avoid incorrect dosage and related complications.

Preparations containing natural thyroid hormones also are available. Prior to the availability of pure levothyroxine, desiccated animal thyroid extract was the only available treatment for hypothyroidism. Some individuals prefer desiccated thyroid extract as a more “natural” thyroid hormone. In addition, some patients who continue to have symptoms of hypothyroidism when taking levothyroxine report improvement in these symptoms when switched to desiccated thyroid extract. Thyroid powder is a natural preparation derived from porcine thyroid glands containing thyroid hormones T3 (liothyronine) and T4 (levothyroxine). As is the case with synthetic thyroid hormones, naturally derived liothyronine and levothyroxine can degrade over time, and natural thyroid hormone replacement preparations have a shelf life set to ensure better that individual dosage forms supply the desired amount of thyroid hormone to a patient. Natural thyroid dosage forms exhibiting enhanced stability of active drug product can reduce the risk of inadequate dosage delivery.

Accordingly, there is a need for a thyroid hormone replacement dosage form including natural thyroid hormones and which exhibits advantageous stability and improved shelf life.

### BRIEF DESCRIPTION OF THE DRAWINGS

Various features and characteristics of non-limiting and non-exhaustive embodiments disclosed and described in this specification may be better understood by reference to the accompanying figures, in which:

FIGS. 1A and 1B, respectively, are side and section views of an embodiment of a 30 cm³ (1.0 oz.) white high-density polyethylene (“HDPE”) bottle used to contain oral solid dosage forms, as described herein.

FIG. 1C is a side view of Detail “A” in FIG. 1A.

FIGS. 2A-2C are views of an embodiment of a container closure system that can be used with the container shown in FIGS. 1A, 1B, and 1C, wherein FIG. 2A is an exterior elevational view of a ribbed, threaded closure cap, FIG. 2B is an exterior top view of the ribbed, threaded closure cap, and FIG. 2C is a side sectional view of the cap shown in FIG. 2B.

FIGS. 3A and 3B, respectively, are side and section views of an embodiment of a 75 cm³ (2.5 oz.) white HDPE bottle used to contain oral solid dosage forms, as described herein.

FIG. 3C is a side view of Detail “A” in FIG. 3A.

FIGS. 4A and 4B are views of an embodiment of a container closure system that can be used with the container shown in FIGS. 3A. 3B, and 3C, wherein FIG. 4A is an exterior top view of a ribbed, threaded closure cap and FIG. 4B is a side sectional view of the cap shown in FIG. 4A.

The reader will appreciate the foregoing details, as well as others, upon considering the following detailed description of various non-limiting and non-exhaustive embodiments according to the present disclosure.

### DESCRIPTION OF CERTAIN NON-LIMITING EMBODIMENTS

Various embodiments are described and illustrated in this specification to provide an overall understanding of the disclosed compositions and methods. It is understood that the various embodiments described and illustrated in this specification are non-limiting and non-exhaustive. Thus, the present invention is not limited by the description of the various non-limiting and non-exhaustive embodiments disclosed in this specification. Rather, the invention is defined solely by the claims. Certain features and characteristics illustrated and/or described in connection with various embodiments may be combined with the features and characteristics of other embodiments. Such modifications and variations are intended to be included within the scope of this specification. As such, the claims may be amended to recite any features or characteristics expressly or inherently described in, or otherwise expressly or inherently supported by, this specification. Further, Applicant reserves the right to amend the claims to affirmatively disclaim features or characteristics that may be found present in the prior art. The various embodiments disclosed and described in this specification can comprise, consist of, or consist essentially of the features and characteristics as variously described herein.

All percentages and ratios provided herein are weight percentages based on the total weight of the respective composition, tablet, mixture, etc., unless otherwise indicated.

Any numerical ranges recited in this specification are intended to include all sub-ranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this specification is intended to include all lower numerical limitations subsumed therein and any minimum numerical limitation recited in this specification is intended to include all higher numerical limitations subsumed therein. Applicant reserves the right to amend this specification, including the claims, to expressly recite any sub-range subsumed within the ranges expressly recited herein.

As generally used herein, the term “about” refers to an acceptable degree of error for the quantity measured, given the nature or precision of the measurement. Typical exemplary degrees of error may be within 20%, 10%, or 5% of a given value or range of values.

The terms “subject” and “patient” are used interchangeably herein, and it is intended that both refer to a recipient on whom a method is conducted according to the present disclosure or another method, as the case may be.

The term “ready to use” as used herein refers to a composition that is suitable for administration to a patient without further preparation.

The term “stability” as used herein refers to each oral solid dosage form retaining at least 90%, at least 95%, at least 99% or at least 100% of an initial levothyroxine content and/or an initial liothyronine content in the oral solid dosage form after the oral solid dosage form has been stored for a period of time.

Disclosed herein are storage stable, oral solid dosage forms. The oral solid dosage forms (e.g., tablet) comprise naturally derived porcine thyroid powder, microcrystalline cellulose and at least one excipient selected from a lubricant, a glidant, a diluent, and a disintegrant. The present inventors observed that embodiments of the oral solid dosage forms according to the present disclosure exhibit unexpectedly advantageous stability of the naturally derived levothyroxine and liothyronine in the drug forms.

The porcine thyroid powder that may be included in the present oral solid dosage forms comprises two active pharmaceutical ingredients: levothyroxine (also known as thyroxine and L-thyroxine) and liothyronine (also known as(S)-triiodothyronine), which are both illustrated below.

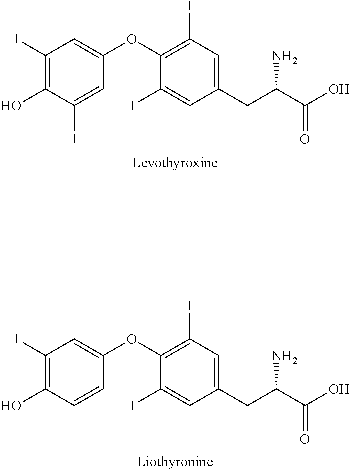 [SMILES: N[C@@H](Cc1cc(I)c(Oc2cc(I)c(O)c(I)c2)c(I)c1)C(=O)O ; N[C@@H](Cc1cc(I)c(Oc2ccc(O)c(I)c2)c(I)c1)C(=O)O]

The amount of porcine thyroid powder included in an oral dosage form according to the present disclosure is selected to provide desired amounts of levothyroxine and liothyronine in the oral solid dosage form.

In certain non-limiting embodiments, porcine thyroid powder may be present in the oral solid dosage forms according to the present disclosure in a concentration of 45% to 55%, in weight percent based on the total weight of the oral solid dosage form, such as, for example, 46% to 55%, 47% to 55%, 48% to 55%, 49% to 55%, 50% to 55%, 46% to 54%, 47% to 54%, 48% to 54%, 49% to 54%, 50% to 54%, 46% to 53%, 47% to 53%, 48% to 53%, 48% to 52%, 49% to 52%, or 50% to 52%, all in weight percent based on total weight of the oral solid dosage form. In other embodiments, the porcine thyroid powder may be present in the oral solid dosage forms in a concentration of about 45%, about 46%, about 47%, about 48%, about 49%, about 50%, about 51%, about 52%, about 53%, about 54%, or about 55%, in weight based on the total weight of the oral solid dosage form.

In certain non-limiting embodiments, depending on the desired strength of the particular oral solid dosage form, the mass of porcine thyroid powder included in an oral solid dosage form according to the present disclosure can range from about 12 mg to about 20 mg, from about 13 mg to about 20 mg, from about 14 mg to about 20 mg, from about 15 mg to about 20 mg, from about 12 mg to about 19 mg, from about 12 mg to about 18 mg, from about 12 mg to about 17 mg, from about 24 mg to about 40 mg, from about 24 mg to about 38 mg, from about 24 mg to about 36 mg, from about 24 mg to about 34 mg, from about 28 mg to about 40 mg, from about 30 mg to about 40 mg, from about 28 mg to about 34 mg, from about 49 mg to about 81 mg, from about 49 mg to about 78 mg, from about 49 mg to about 72 mg, from about 49 mg to about 68 mg, from about 52 mg to about 78 mg, from about 56 mg to about 74 mg, from about 60 mg to about 68 mg, from about 73 mg to about 122 mg, from about 73 mg to about 114 mg, from about 73 mg to about 106 mg, from about 80 mg to about 116 mg, from about 86 mg to about 110 mg, from about 90 mg to about 102 mg, from about 94 mg to about 100 mg, from about 96 mg to about 162 mg, from about 100 mg to about 162 mg, from about 110 mg to about 162 mg, from about 110 mg to about 150 mg, from about 120 mg to about 150 mg, from about 125 mg to about 140 mg, from about 175 mg to about 215 mg, from about 185 mg to about 215 mg, from about 190 mg to about 215 mg, from about 175 mg to about 210 mg, from about 175 mg to about 200 mg, from about 190 mg to about 200 mg, from about 230 mg to about 290 mg, from about 240 mg to about 290 mg, from about 250 mg to about 290 mg, from about 230 mg to about 280 mg, from about 230 mg to about 270 mg, from about 250 mg to about 270 mg, from about 255 mg to about 265 mg, from about 290 mg to about 360 mg, from about 300 mg to about 360 mg, from about 310 mg to about 360 mg, from about 290 mg to about 350 mg, from about 290 mg to about 340 mg, from about 310 mg to about 340 mg, or from about 320 mg to about 330 mg.

Various embodiments of oral solid dosage forms according to the present disclosure include an amount of porcine thyroid powder comprising 7 μg to 230 μg of levothyroxine. Various embodiments of oral solid dosage forms according to the present disclosure include an amount of porcine thyroid powder comprising 1.6 μg to 55 μg of liothyronine. Various embodiments of oral solid dosage forms according to the present disclosure include an amount of porcine thyroid powder comprising 7 μg to 230 μg of levothyroxine and 1.6 μg to 55 μg of liothyronine.

In various non-limiting embodiments, an oral solid dosage form according to the present disclosure may comprise an amount of porcine thyroid powder comprising 7.0 μg to 11.9 μg of levothyroxine and 1.6 μg to 2.8 μg of liothyronine. In various other non-limiting embodiments, an oral solid dosage form according to the present disclosure may comprise an amount of porcine thyroid powder comprising 14.2 μg to 23.8 μg of levothyroxine and 3.3 μg to 5.6 μg of liothyronine. In various other non-limiting embodiments, an oral solid dosage form according to the present disclosure may comprise an amount of porcine thyroid powder comprising 28.5 μg to 47.5 μg of levothyroxine and 6.7 μg to 11.3 μg of liothyronine. In various other non-limiting embodiments, an oral solid dosage form according to the present disclosure may comprise an amount of porcine thyroid powder comprising 42.7 μg to 71.3 μg of levothyroxine and 10.1 μg to 16.9 μg of liothyronine. In various other non-limiting embodiments, an oral solid dosage form according to the present disclosure may comprise an amount of porcine thyroid powder comprising 57 μg to 95 μg of levothyroxine and 13.5 μg to 22.5 μg of liothyronine. In various other non-limiting embodiments, an oral solid dosage form according to the present disclosure may comprise an amount of porcine thyroid powder comprising 91 μg to 139 μg of levothyroxine and 21 μg to 33 μg of liothyronine. In various other non-limiting embodiments, an oral solid dosage form according to the present disclosure may comprise an amount of porcine thyroid powder comprising 121 μg to 183 μg of levothyroxine and 28 μg to 44 μg of liothyronine. In various other non-limiting embodiments, an oral solid dosage form according to the present disclosure may comprise an amount of porcine thyroid powder comprising 152 μg to 228 μg of levothyroxine and 36 μg to 54 μg of liothyronine.

Various embodiments of an oral solid dosage form according to the present disclosure can comprise one or more diluents. Diluents may be included to act as fillers in the oral solid dosage form (e.g., tablet) to increase dosage form weight and improve content uniformity. Non-limiting examples of suitable diluents that can be used in the oral solid dosage forms disclosed herein include at least one of microcrystalline cellulose (MCC), AVICEL® HFE-102 powder (a blend of microcrystalline cellulose and mannitol, available from FMC BioPolymer, Philadelphia, PA), confectioner's sugar (including corn starch), croscarmellose sodium, dicalcium phosphate, inulin, carbohydrates such as, for example, arabinose, sucrose, dextrose, fructose, maltose, lactose, lactose monohydrate, trehalose, isomalt, starch, monosaccharides, disaccharides, polysaccharides, sugar alcohols (e.g., sorbitol, mannitol, erythritol, xylitol, lactitol), derivatives of the foregoing, and combinations of any thereof.

In certain non-limiting embodiments, diluent may be present in oral solid dosage forms according to the present disclosure in a concentration of 35% to 49%, in weight percent based on the total weight of the oral solid dosage form, such as, for example, 35% to 48%, 35% to 47%, 35% to 46%, 35% to 45%, 38% to 48%, 38% to 47%, 38% to 46%, 38% to 45%, 40% to 49%, 40% to 48, 40% to 47%, 40% to 46%, 40% to 45%, 41% to 49%, 41% to 48%, 41% to 47%, 41% to 46%, 41% to 45%, 42% to 49%, 42% to 48%, 42% to 47%, 42% to 46%, 42% to 45%, 43% to 49%, 43% to 48%, 43% to 47%, 43% to 46%, or 43% to 45%. In other embodiments, diluent may be present in the oral solid dosage forms according to the present disclosure in a concentration of about 35%, about 36%, about 37%, about 38%, about 39%, about 40%, about 41%, about 42%, about 43%, about 44%, or about 45%, all in weight percent based on the total weight of the oral solid dosage form.

In certain non-limiting embodiments, the amount of diluent included in an oral solid dosage form can range from about 10.5 mg to about 17.5 mg, from about 11.5 mg to about 17.5 mg, from about 12.5 mg to about 17.5 mg, from about 13.5 mg to about 17.5 mg, from about 10.5 mg to about 16.5 mg, from about 10.5 mg to about 15.5 mg, from about 10.5 mg to about 14.5 mg, from about 21 mg to about 36 mg, from about 21 mg to about 34 mg, from about 21 mg to about 32 mg, from about 21 mg to about 30 mg, from about 24 mg to about 32 mg, from about 26 mg to about 34 mg, from about 26 mg to about 30 mg, from about 43 mg to about 71 mg, from about 43 mg to about 68 mg, from about 43 mg to about 64 mg, from about 43 mg to about 60 mg, from about 46 mg to about 71 mg, from about 50 mg to about 66 mg, from about 52 mg to about 64 mg, from about 65 mg to about 107 mg, from about 72 mg to about 107 mg, from about 78 mg to about 107 mg, from about 82 mg to about 102 mg, from about 72 mg to about 102 mg, from about 76 mg to about 100 mg, from about 82 mg to about 90 mg, from about 85 mg to about 140 mg, from about 85 mg to about 130 mg, from about 85 mg to about 125 mg, from about 85 mg to about 120 mg, from about 90 mg to about 140 mg, from about 95 mg to about 140 mg, from about 100 mg to about 130 mg, from about 105 mg to about 120 mg, from about 155 mg to about 190 mg, from about 155 mg to about 180 mg, from about 165 mg to about 190 mg, from about 165 mg to about 180 mg, from about 165 mg to about 175 mg, from about 205 mg to about 255 mg, from about 205 mg to about 245 mg, from about 205 mg to about 235 mg, from about 215 mg to about 255 mg, from about 225 mg to about 255 mg, from about 220 mg to about 240 mg, from about 225 mg to about 235 mg, from about 255 mg to about 320 mg, from about 265 mg to about 320 mg, from about 275 mg to about 320 mg, from about 255 mg to about 310 mg, from about 255 mg to about 300 mg, from about 265 mg to about 300 mg, or from about 275 mg to about 295 mg.

In certain non-limiting embodiments, microcrystalline cellulose (MCC) may be present in the oral solid dosage forms according to the present disclosure in a concentration of 35% to 49% weight percent based on the total weight of the oral solid dosage form, such as, for example, 35% to 48%, 35% to 47%, 35% to 46%, 35% to 45%, 38% to 48%, 38% to 47%, 38% to 46%, 38% to 45%, 40% to 49%, 40% to 48, 40% to 47%, 40% to 46%, 40% to 45%, 41% to 49%, 41% to 48%, 41% to 47%, 41% to 46%, 41% to 45%, 42% to 49%, 42% to 48%, 42% to 47%, 42% to 46%, 42% to 45%, 43% to 49%, 43% to 48%, 43% to 47%, 43% to 46%, or 43% to 45%, all in weight percent based on the total weight of the oral solid dosage form. In other embodiments, MCC may be present in the oral solid dosage forms according to the present disclosure in a concentration of about 35%, about 36%, about 37%, about 38%, about 39%, about 40%, about 41%, about 42%, about 43%, about 44%, or about 45%, all in weight percent based on the total weight of the oral solid dosage form.

In certain non-limiting embodiments, the amount of microcrystalline cellulose included in an oral solid dosage form according to the present disclosure can range from about 10.5 mg to about 17.5 mg, from about 11.5 mg to about 17.5 mg, from about 12.5 mg to about 17.5 mg, from about 13.5 mg to about 17.5 mg, from about 10.5 mg to about 16.5 mg, from about 10.5 mg to about 15.5 mg, from about 10.5 mg to about 14.5 mg, from about 21 mg to about 36 mg, from about 21 mg to about 34 mg, from about 21 mg to about 32 mg, from about 21 mg to about 30 mg, from about 24 mg to about 32 mg, from about 26 mg to about 34 mg, from about 26 mg to about 30 mg, from about 43 mg to about 71 mg, from about 43 mg to about 68 mg, from about 43 mg to about 64 mg, from about 43 mg to about 60 mg, from about 46 mg to about 71 mg, from about 50 mg to about 66 mg, from about 52 mg to about 64 mg, from about 65 mg to about 107 mg, from about 72 mg to about 107 mg, from about 78 mg to about 107 mg, from about 82 mg to about 102 mg, from about 72 mg to about 102 mg, from about 76 mg to about 100 mg, from about 82 mg to about 90 mg, from about 85 mg to about 140 mg, from about 85 mg to about 130 mg, from about 85 mg to about 125 mg, from about 85 mg to about 120 mg, from about 90 mg to about 140 mg, from about 95 mg to about 140 mg, from about 100 mg to about 130 mg, from about 105 mg to about 120 mg, from about 155 mg to about 190 mg, from about 155 mg to about 180 mg, from about 165 mg to about 190 mg, from about 165 mg to about 180 mg, from about 165 mg to about 175 mg, from about 205 mg to about 255 mg, from about 205 mg to about 245 mg, from about 205 mg to about 235 mg, from about 215 mg to about 255 mg, from about 225 mg to about 255 mg, from about 220 mg to about 240 mg, from about 225 mg to about 235 mg, from about 255 mg to about 320 mg, from about 265 mg to about 320 mg, from about 275 mg to about 320 mg, from about 255 mg to about 310 mg, from about 255 mg to about 300 mg, from about 265 mg to about 300 mg, or from about 275 mg to about 295 mg.

AVICEL® HFE-102 powder is a pharmaceutical diluent that is a spray dried blend of about 90 weight percent microcrystalline cellulose and about 10 weight percent mannitol. In certain non-limiting embodiments, AVICEL® HFE-102 powder may be present in embodiments of oral solid dosage forms according to the present disclosure in a concentration of 35% to 49%, in weight percent based on the total weight of the oral solid dosage form, such as, for example, 35% to 48%, 35% to 47%, 35% to 46%, 35% to 45%, 38% to 48%, 38% to 47%, 38% to 46%, 38% to 45%, 40% to 49%, 40% to 48, 40% to 47%, 40% to 46%, 40% to 45%, 41% to 49%, 41% to 48%, 41% to 47%, 41% to 46%, 41% to 45%, 42% to 49%, 42% to 48%, 42% to 47%, 42% to 46%, 42% to 45%, 43% to 49%, 43% to 48%, 43% to 47%, 43% to 46%, and 43% to 45%, all in weight percent based on the total weight of the oral solid dosage form. In certain non-limiting embodiments, AVICEL® HFE-102 powder may be present in the oral solid dosage forms according to the present disclosure in a concentration of about 35%, about 36%, about 37%, about 38%, about 39%, about 40%, about 41%, about 42%, about 43%, about 44%, or about 45%, all in weight percent based on the total weight of the oral solid dosage form.

In certain non-limiting embodiments, the amount of AVICEL® HFE-102 powder included in an oral solid dosage form according to the present disclosure can range from about 10.5 mg to about 17.5 mg, from about 11.5 mg to about 17.5 mg, from about 12.5 mg to about 17.5 mg, from about 13.5 mg to about 17.5 mg, from about 10.5 mg to about 16.5 mg, from about 10.5 mg to about 15.5 mg, from about 10.5 mg to about 14.5 mg, from about 21 mg to about 36 mg, from about 21 mg to about 34 mg, from about 21 mg to about 32 mg, from about 21 mg to about 30 mg, from about 24 mg to about 32 mg, from about 26 mg to about 34 mg, from about 26 mg to about 30 mg, from about 43 mg to about 71 mg, from about 43 mg to about 68 mg, from about 43 mg to about 64 mg, from about 43 mg to about 60 mg, from about 46 mg to about 71 mg, from about 50 mg to about 66 mg, from about 52 mg to about 64 mg, from about 65 mg to about 107 mg, from about 72 mg to about 107 mg, from about 78 mg to about 107 mg, from about 82 mg to about 102 mg, from about 72 mg to about 102 mg, from about 76 mg to about 100 mg, from about 82 mg to about 90 mg, from about 85 mg to about 140 mg, from about 85 mg to about 130 mg, from about 85 mg to about 125 mg, from about 85 mg to about 120 mg, from about 90 mg to about 140 mg, from about 95 mg to about 140 mg, from about 100 mg to about 130 mg, from about 105 mg to about 120 mg, from about 155 mg to about 190 mg, from about 155 mg to about 180 mg, from about 165 mg to about 190 mg, from about 165 mg to about 180 mg, from about 165 mg to about 175 mg, from about 205 mg to about 255 mg, from about 205 mg to about 245 mg, from about 205 mg to about 235 mg, from about 215 mg to about 255 mg, from about 225 mg to about 255 mg, from about 220 mg to about 240 mg, from about 225 mg to about 235 mg, from about 255 mg to about 320 mg, from about 265 mg to about 320 mg, from about 275 mg to about 320 mg, from about 255 mg to about 310 mg, from about 255 mg to about 300 mg, from about 265 mg to about 300 mg, or from about 275 mg to about 295 mg.

Various non-limiting embodiments of an oral solid dosage form according to the present disclosure comprise one or more lubricants. Lubricants may be included in the oral solid dosage forms to improve powder processing properties of the formulation when manufacturing the solid dosage forms. Non-limiting examples of suitable lubricants that can be used in the oral solid dosage forms disclosed herein include sodium stearyl fumarate, polyethylene glycols, mineral oil, medium chain triglycerides, sodium stearyl sulfate, cocoa butter, sodium benzoate, stearic acid (and its derivatives or esters such as, for example, sodium stearate, magnesium stearate, calcium stearate), and combinations of any thereof.

The concentration of lubricant in an oral solid dosage form according to certain non-limiting embodiments of the present disclosure can range from about 0.6% to about 1%, from about 0.65% to about 1%, from about 0.6% to about 0.95%, from about 0.7% to about 1%, from about 0.7% to about 0.95%, from about 0.75% to about 1%, from about 0.75% to about 0.95%, from about 0.75% to about 0.95%, from about 0.75% to about 0.90%, from about 0.7% to about 0.95%, from about 0.7% to about 0.85%, or from about 0.75% to about 0.85%, all in weight percent based on total weight of the oral solid dosage form.

In certain non-limiting embodiments, the amount of lubricant included in an oral solid dosage form according to the present disclosure can range from about 0.20 mg to about 0.32 mg, from about 0.21 mg to about 0.32 mg, from about 0.22 mg to about 0.32 mg, from about 0.23 mg to about 0.32 mg, from about 0.24 mg to about 0.32 mg, from about 0.20 mg to about 0.30 mg, from about 0.20 mg to about 0.28 mg, from about 0.40 mg to about 0.64 mg, from about 0.42 mg to about 0.64 mg, from about 0.44 mg to about 0.64 mg, from about 0.46 mg to about 0.64 mg, from about 0.48 mg to about 0.64 mg, from about 0.4 mg to about 0.60 mg, from about 0.40 mg to about 0.56 mg, from about 0.48 mg to about 0.56 mg; from about 0.80 mg to about 1.28 mg, from about 0.84 mg to about 1.28 mg, from about 0.88 mg to about 1.28 mg, from about 0.92 mg to about 1.28 mg, from about 0.96 mg to about 1.28 mg, from about 0.8 mg to about 1.20 mg, from about 0.80 mg to about 1.12 mg, from about 0.96 mg to about 1.12 mg; from about 1.2 mg to about 2.0 mg, from about 1.2 mg to about 1.85 mg, from about 1.2 mg to about 1.7 mg, from about 1.2 mg to about 1.65 mg, from about 1.3 mg to about 1.65 mg, from about 1.4 mg to about 1.65 mg, from about 1.45 mg to about 1.70 mg, from about 1.5 mg to about 1.65 mg; from about 1.5 mg to about 2.6 mg, from about 1.6 mg to about 2.6 mg, from about 1.7 mg to about 2.6 mg, from about 1.8 mg to about 2.6 mg, from about 1.8 mg to about 2.5 mg, from about 1.8 mg to about 2.4 mg, from about 1.8 mg to about 2.3 mg, from about 1.8 mg to about 2.20 mg, from about 2.0 mg to about 2.20 mg, from about 2.75 mg to about 4.0 mg, from about 2.75 mg to about 3.75 mg, from about 2.75 mg to about 3.5 mg, from about 3.0 mg to about 3.5 mg, from about 3.25 mg to about 3.75 mg, from about 3.5 mg to about 5.5 mg, from about 3.75 mg to about 5.5 mg, from about 4.0 mg to about 5.0 mg, from about 4.0 mg to about 4.75 mg, from about 4.5 mg to about 6.0 mg, from about 5.0 mg to about 6.0 mg, from about 5.0 mg to about 5.75 mg, or from about 5.0 mg to about 5.5 mg.

Various non-limiting embodiments of an oral solid dosage form according to the present disclosure can comprise one or more glidants. Glidants may be included in the oral solid dosage forms to enhance flowability of the powder ingredients by reducing interparticle friction, particle surface charge, and/or particle cohesion. Non-limiting examples of suitable glidants that can be used in oral solid dosage forms according to the present disclosure include talc, silicon dioxide, colloidal silicon dioxide, and combinations of any thereof.

The concentration of glidant in an oral solid dosage form according to certain non-limiting embodiments of the present disclosure can range from about 2.0% to about 3%, from about 2.1% to about 3%, from about 2.2% to about 3%, from about 2.3% to about 3%, from about 2.4% to about 3%, from about 2.0% to about 2.9%, from about 2.0% to about 2.8%, from about 2.0% to about 2.7%, from about 2.0% to about 2.6%, from about 2.1% to about 2.6%, from about 2.2% to about 2.6%, from about 2.3% to about 2.6%, all in weight percentages based on total weight of the oral solid dosage form.

In certain non-limiting embodiments, the amount of glidant included in non-limiting embodiments of an oral solid dosage form according to the present disclosure can range from about 0.6 mg to about 1.0 mg, from about 0.64 mg to about 1.0 mg, from about 0.68 mg to about 1.0 mg, from about 0.72 mg to about 1.0 mg, from about 0.76 mg to about 1.0 mg, from about 0.6 mg to about 0.96 mg, from about 0.6 mg to about 0.92 mg, from about 0.6 mg to about 0.88 mg, from about 0.6 mg to about 0.85 mg, from about 0.72 mg to about 0.88 mg, from about 0.76 mg to about 0.85 mg, from about 1.2 mg to about 2.0 mg, from about 1.28 mg to about 2.0 mg, from about 1.36 mg to about 2.0 mg, from about 1.44 mg to about 2.0 mg, from about 1.52 mg to about 2.0 mg, from about 1.2 mg to about 1.96 mg, from about 1.2 mg to about 1.84 mg, from about 1.2 mg to about 1.76 mg, from about 1.2 mg to about 1.68 mg, from about 1.44 mg to about 1.76 mg, from about 1.52 mg to about 1.68 mg, from about 2.4 mg to about 4.0 mg, from about 2.56 mg to about 4.0 mg, from about 2.72 mg to about 4.0 mg, from about 2.88 mg to about 4.0 mg, from about 3.04 mg to about 4.0 mg, from about 2.4 mg to about 3.92 mg, from about 2.4 mg to about 3.68 mg, from about 2.4 mg to about 3.52 mg, from about 2.4 mg to about 3.36 mg, from about 2.88 mg to about 3.52 mg, from about 3.04 mg to about 3.36 mg, from about 3.6 mg to about 6.1 mg, from about 3.6 mg to about 5.8 mg, from about 3.6 mg to about 5.4 mg, from about 3.6 mg to about 5.0 mg, from about 4.0 mg to about 5.8 mg, from about 4.2 mg to about 5.4 mg, from about 4.4 mg to about 5.4 mg, from about 4.6 mg to about 5.2 mg, from about 4.4 mg to about 5.0 mg, from about 4.6 mg to about 5.0 mg, from about 4.8 mg to about 5.0 mg, from about 4.8 mg to about 8.0 mg, from about 5.2 mg to about 8.0 mg, from about 5.6 mg to about 8.0 mg, from about 6.0 mg to about 8.0 mg, from about 4.8 mg to about 7.2 mg, from about 4.8 mg to about 7.2 mg, from about 5.2 mg to about 7.6 mg, from about 5.6 mg to about 7.6 mg, from about 5.6 mg to about 7.2 mg, from about 5.6 mg to about 6.8 mg, from about 6.0 mg to about 6.8 mg, from about 7.0 mg to about 11 mg, from about 8.0 mg to about 11 mg, from about 9 mg to about 11 mg, from about 9.5 mg to about 11 mg, from about 9.5 mg to about 10.5 mg, from about 11.0 mg to about 14 mg, from about 12 mg to about 14 mg, from about 12.5 mg to about 14 mg, from about 13 mg to about 14 mg, from about 12.5 mg to about 13.5 mg, from about 14.0 mg to about 17.0 mg, from about 15.0 mg to about 17.0 mg, from about 15.0 mg to about 17.0 mg, from about 16.0 mg to about 17.0 mg, from about 16.0 mg to about 16.75 mg.

Various non-limiting embodiments of an oral solid dosage form according to the present disclosure can comprise one or more disintegrant. Disintegrants may be included in the oral solid dosage forms to facilitate disintegration after oral administration. Non-limiting examples of suitable disintegrants that can be used in oral solid dosage forms according to the present disclosure include sodium starch glycolate, croscarmellose sodium, and combinations of any thereof.

The concentration of disintegrant in an oral solid dosage form according to certain non-limiting embodiments of the present disclosure can range from about 2.0% to about 3%, from about 2.1% to about 3%, from about 2.2% to about 3%, from about 2.3% to about 3%, from about 2.4% to about 3%, from about 2.0% to about 2.9%, from about 2.0% to about 2.8%, from about 2.0% to about 2.7%, from about 2.0% to about 2.6%, from about 2.1% to about 2.6%, from about 2.2% to about 2.6%, or from about 2.3% to about 2.6%, all in weight percentages based on total weight of the oral solid dosage form.

In certain non-limiting embodiments, the amount of disintegrant included in an oral solid dosage form according to the present disclosure can range from about 0.6 mg to about 1.0 mg, from about 0.64 mg to about 1.0 mg, from about 0.68 mg to about 1.0 mg, from about 0.72 mg to about 1.0 mg, from about 0.76 mg to about 1.0 mg, from about 0.6 mg to about 0.96 mg, from about 0.6 mg to about 0.92 mg, from about 0.6 mg to about 0.88 mg, from about 0.6 mg to about 0.85 mg, from about 0.72 mg to about 0.88 mg, from about 0.76 mg to about 0.85 mg, from about 1.2 mg to about 2.0 mg, from about 1.28 mg to about 2.0 mg, from about 1.36 mg to about 2.0 mg, from about 1.44 mg to about 2.0 mg, from about 1.52 mg to about 2.0 mg, from about 1.2 mg to about 1.96 mg, from about 1.2 mg to about 1.84 mg, from about 1.2 mg to about 1.76 mg, from about 1.2 mg to about 1.68 mg, from about 1.44 mg to about 1.76 mg, from about 1.52 mg to about 1.68 mg, from about 2.4 mg to about 4.0 mg, from about 2.56 mg to about 4.0 mg, from about 2.72 mg to about 4.0 mg, from about 2.88 mg to about 4.0 mg, from about 3.04 mg to about 4.0 mg, from about 2.4 mg to about 3.92 mg, from about 2.4 mg to about 3.68 mg, from about 2.4 mg to about 3.52 mg, from about 2.4 mg to about 3.36 mg, from about 2.88 mg to about 3.52 mg, from about 3.04 mg to about 3.36 mg, from about 3.6 mg to about 6.1 mg, from about 3.6 mg to about 5.8 mg, from about 3.6 mg to about 5.4 mg, from about 3.6 mg to about 5.0 mg, from about 4.0 mg to about 5.8 mg, from about 4.2 mg to about 5.4 mg, from about 4.4 mg to about 5.4 mg, from about 4.6 mg to about 5.2 mg, from about 4.4 mg to about 5.0 mg, from about 4.6 mg to about 5.0 mg, from about 4.8 mg to about 5.0 mg, from about 4.8 mg to about 8.0 mg, from about 5.2 mg to about 8.0 mg, from about 5.6 mg to about 8.0 mg, from about 6.0 mg to about 8.0 mg, from about 4.8 mg to about 7.2 mg, from about 4.8 mg to about 7.2 mg, from about 5.2 mg to about 7.6 mg, from about 5.6 mg to about 7.6 mg, from about 5.6 mg to about 7.2 mg, from about 5.6 mg to about 6.8 mg, from about 6.0 mg to about 6.8 mg, from about 7.0 mg to about 11 mg, from about 8.0 mg to about 11 mg, from about 9 mg to about 11 mg, from about 9.5 mg to about 11 mg, from about 9.5 mg to about 10.5 mg, from about 11.0 mg to about 14 mg, from about 12 mg to about 14 mg, from about 12.5 mg to about 14 mg, from about 13 mg to about 14 mg, from about 12.5 mg to about 13.5 mg, from about 14.0 mg to about 17.0 mg, from about 15.0 mg to about 17.0 mg, from about 15.0 mg to about 17.0 mg, from about 16.0 mg to about 17.0 mg, from about 16.0 mg to about 16.75 mg.

Various non-limiting embodiments of an oral solid dosage form according to the present disclosure can further comprise one or more coatings. Coatings may be included on the oral solid dosage forms to, for example, mask taste, facilitate swallowing, and/or protect active pharmaceutical ingredients. Non-limiting examples of suitable coatings that can be applied to various non-limiting embodiments of the oral solid dosage forms according to the present disclosure include OPADRY® white, OPADRY® II 85F19316 clear, and combinations of any thereof.

In various non-limiting embodiments, the oral solid dosage forms according to the present disclosure may be provided as tablets, caplets, capsules, powders, lozenges, or other suitable solid dosage forms.

In certain embodiments according to the present disclosure, an immediate release oral solid dosage form comprises porcine thyroid powder, microcrystalline cellulose, and at least one excipient selected from a lubricant, a glidant, a diluent, and a disintegrant. In various embodiments, the immediate release oral solid dosage form is prepared by a method comprising a dry blending process. In various embodiments, the immediate release oral solid dosage form retains at least 90% of an initial levothyroxine content in the solid dosage form when the dosage form is stored for twenty-four months under standard testing conditions (25° C.±2° C. and 60%±5% relative humidity) and/or 6 months under intermediate conditions (30° C.±2° C. and 65%±5% relative humidity) and/or 6 months under accelerated testing conditions (40° C.±2° C. and 75%±5% relative humidity).

In certain embodiments according to the present disclosure, an immediate release oral solid dosage form comprises porcine thyroid powder, microcrystalline cellulose, and at least one excipient selected from a lubricant, a glidant, a diluent, and a disintegrant. In various embodiments, the immediate release oral solid dosage form is prepared by a method comprising a dry blending process. In various embodiments, the immediate release oral solid dosage form retains at least 90% of an initial liothyronine content in the solid dosage form when the dosage form is stored for twenty-four months under either standard testing conditions (25° C.±2° C. and 60%+5% relative humidity), and/or 6 months under intermediate conditions (30° C.±2° C. and 65%±5% relative humidity), and/or 6 months under accelerated testing conditions (40° C.±2° C. and 75%±5% relative humidity).

In certain embodiments according to the present disclosure, an immediate release oral solid dosage form comprises a porcine thyroid powder, microcrystalline cellulose, and at least one excipient selected from a lubricant, a glidant, a diluent, and a disintegrant. In various non-limiting embodiments, the immediate release oral solid dosage form is prepared by a method comprising a dry blending process. In various embodiments, the immediate release oral solid dosage form retains at least 95% of an initial levothyroxine content in the solid dosage form when the dosage form is stored for twenty-four months under standard testing conditions (25° C.±2° C. and 60%±5% relative humidity), and/or 6 months under intermediate conditions (30° C.±2° C. and 65%±5% relative humidity), and/or 6 months under accelerated testing conditions (40° C.±2° C. and 75%±5% relative humidity).

In certain embodiments according to the present disclosure, an immediate release oral solid dosage form comprises a porcine thyroid powder, microcrystalline cellulose, and at least one excipient selected from a lubricant, a glidant, a diluent, and a disintegrant. In various non-limiting embodiments, the immediate release oral solid dosage form is prepared by a method comprising a dry blending process. In various embodiments, the immediate release oral solid dosage form retains at least 95% of an initial liothyronine content in the solid dosage form when the dosage form is stored for twenty-four months under standard testing conditions (25° C.±2° C. and 60%±5% relative humidity), and/or 6 months under intermediate conditions (30° C.±2° C. and 65%±5% relative humidity), and/or 6 months under accelerated testing conditions (40° C.±2° C. and 75%±5% relative humidity).

In certain embodiments according to the present disclosure, an immediate release oral solid dosage form prepared by a dry uniform blending process comprises a porcine thyroid powder, microcrystalline cellulose, and at least one excipient selected from a lubricant, a glidant, a diluent, and a disintegrant. In various non-limiting embodiments, the immediate release oral solid dosage form is prepared by a method comprising a dry blending process. In various embodiments, the immediate release oral solid dosage form retains at least 99% of an initial levothyroxine content in the solid dosage form when the dosage form is stored for twenty-four months under standard testing conditions (25° C.±2° C. and 60%±5% relative humidity), and/or 6 months under intermediate conditions (30° C. #2° C. and 65% #5% relative humidity), and/or 6 months under accelerated testing conditions (40° C.±2° C. and 75%±5% relative humidity).

In certain embodiments according to the present disclosure, an immediate release oral solid dosage form prepared by a dry uniform blending process comprises a porcine thyroid powder, microcrystalline cellulose, and at least one excipient selected from a lubricant, a glidant, a diluent, and a disintegrant. In various non-limiting embodiments, the immediate release oral solid dosage form is prepared by a method comprising a dry blending process. In various embodiments, the immediate release oral solid dosage form retains at least 99% of an initial liothyronine content in the solid dosage form when the dosage form is stored for twenty-four months under standard testing conditions (25° C.±2° C. and 60%±5% relative humidity), and/or 6 months under intermediate conditions (30° C.±2° C. and 65%±5% relative humidity), and/or 6 months under accelerated testing conditions (40° C.±2° C. and 75%±5% relative humidity).

In various embodiments, immediate release oral solid dosage forms according to the present disclosure can be prepared by a method comprising a dry blending process that provides for uniform blending of ingredients. The dry uniform blending of the ingredients can comprise techniques known in the art, such as one or more of blending, mixing, sieving, co-milling, grinding, granulation, and power granulation.

In certain non-limiting embodiments, a prepackaged drug product is provided comprising oral solid dosage forms according to the present disclosure contained in a container closure system including a container and a sealing closure. In certain non-limiting embodiments, the container has an internal volume of about 15 cm³ to about 45 cm³ and includes therein about 5 to about 15 oral solid dosage forms according to the present disclosure and, optionally, at least one silica gel desiccant canister containing, for example, about 2 grams of desiccant. In various embodiments, the container closure system can also include at least one oxygen absorber containing, for example, about 2 grams of oxygen absorbing material. In certain non-limiting embodiments, a prepackaged drug product according to the present disclosure comprises a container closure system wherein the container has an internal volume of about 15 cm³ to about 45 cm³ and in which are contained about 5 to about 15 oral solid dosage forms according to the present disclosure, at least one silica gel desiccant canister (including, for example, about 2 grams of desiccant), and at least one oxygen absorber (including, for example, about 2 grams of oxygen absorbing material).

In other non-limiting embodiments, a prepackaged drug product is provided comprising oral solid dosage forms according to the present disclosure contained in a container closure system including a container and a sealing closure. In certain non-limiting embodiments, the container has an internal volume of about 60 cm³ to about 90 cm³ and includes therein about 80 to about 100 oral solid dosage forms according to the present disclosure and, optionally, at least one silica gel desiccant canister containing, for example, about 2 grams of desiccant. In various embodiments, the container closure system can also include therein at least one oxygen absorber containing, for example, about 2 grams of oxygen absorbing material. In certain non-limiting embodiments, a prepackaged drug product according to the present disclosure comprises a container closure system wherein the container has an internal volume of about 60 cm³ to about 90 cm³ and in which are contained about 80 to about 100 oral solid dosage forms according to the present disclosure, at least one silica gel desiccant canister (including, for example, about 2 grams of desiccant), and at least one oxygen absorber (including, for example, about 2 grams of oxygen absorbing material).

The container of the container closure system of certain non-limiting embodiments of a prepackaged drug product according to the present disclosure can comprise at least one of glass, plastic, or other suitable polymer material. For example, in certain embodiments the container of the container closure system of certain non-limiting embodiments of a prepackaged drug product according to the present disclosure can comprise polypropylene or high-density polyethylene. In certain embodiments of a prepackaged drug product according to the present disclosure, the oral solid dosage forms can be stored in a container of container closure system that has been purged with an inert gas to replace at least a portion of environmental oxygen within the container prior to sealing the container with the closure. In certain embodiments, the inert gas used to purge the container can comprise nitrogen, argon, a mixture of nitrogen and argon, or another inert gas. In certain embodiments, at least a portion of air space within the container may be filled with a pill packing material such as, for example, rayon or cotton, prior to sealing the container with the closure.

An aspect of the present disclosure is directed to methods of administering an oral solid dosage form comprising thyroid hormone according to the present disclosure to a patient or human subject in need thereof. According to one method, an immediate release oral solid dosage form according to the present disclosure is administered to a patient or human subject in need thereof such that that the patient/subject receives about 7 μg to about 230 μg of levothyroxine and about 1.6 μg to about 55 μg of liothyronine per dosage.

According to certain non-limiting embodiments, oral solid dosage forms according to the present disclosure may be prepared using a dry layering/mixing process generally including the following steps. A dry layered blend is prepared by first screening a diluent (e.g., AVICEL® HFE-102 powder or MCC) through a #30 mesh screen and then mixing the diluent using a V-blender to form a first layer in the V-blender. Next, a screened blend is prepared by screening together the remaining diluent, a disintegrant (e.g., sodium starch glycolate), and a glidant (e.g., colloidal silicon dioxide) to form a screened blend. The screened blend is deposited on the first layer to form a second layer in the V-blender. Porcine thyroid powder is deposited on the second layer to form a third layer in the V-blender. Next, a fourth layer is formed on the third layer by screening a lubricant (e.g., calcium stearate) through a #30 mesh screen and depositing it on the third layer in the V-blender. The dry layered arrangement comprising the first, second, third, and fourth layers of powdered materials is then uniformly mixed in the V-blender to provide a uniform powder mixture. Predetermined quantities of the dry mixture are compressed to form tablets having a desired mass and including desired quantities of levothyroxine and liothyronine per tablet. Alternatively, predetermined quantities of the dry oral solid dosage mixture may be, for example, disposed in capsules such as, for example, hard gelatin capsules, or used to prepare other suitable oral solid dosage forms.

The present inventors observed that embodiments of oral solid dosage forms according to the present disclosure including porcine thyroid powder exhibit advantageous stability. For example, the present inventors observed that certain non-limiting embodiments of oral solid dosage forms according to the present disclosure retained at least 90% of an initial amount of levothyroxine after storage for three months, four months, five months, six months, nine months, twelve months, eighteen months, or twenty-four months under standard testing conditions including a temperature of about 23° C. to about 27° C. and a relative humidity ranging from about 55% to about 65%. The present inventors observed that certain non-limiting embodiments of oral solid dosage forms according to the present disclosure retained at least 90% of an initial amount of levothyroxine after storage for three months or six months under intermediate testing conditions including a temperature of about 28° C. to about 32° C. and a relative humidity ranging from about 60% to about 70%. The present inventors also observed that certain embodiments of oral solid dosage forms according to the present disclosure retained at least 90% of an initial amount of levothyroxine after storage for one month, two months, three months, four months, five months, or six months under accelerated testing conditions including a temperature of about 38° C. to about 42° C. and a relative humidity ranging from about 70% to about 80%.

The present inventors observed that certain non-limiting embodiments of oral solid dosage forms according to the present disclosure retained at least 92% of an initial amount of levothyroxine after storage for three months, four months, five months, six months, nine months, twelve months, eighteen months, or twenty-four months under standard testing conditions including a temperature of about 23° C. to about 27° C. and a relative humidity ranging from about 55% to about 65%. The present inventors observed that certain non-limiting embodiments of oral solid dosage forms according to the present disclosure retained at least 92% of an initial amount of levothyroxine after storage for three months or six months under intermediate testing conditions including a temperature of about 28° C. to about 32° C. and a relative humidity ranging from about 60% to about 70%. The present inventors also observed that certain embodiments of oral solid dosage forms according to the present disclosure retained at least 92% of an initial amount of levothyroxine after storage for one month, two months, three months, four months, five months, or six months under accelerated testing conditions including a temperature of about 38° C. to about 42° C. and a relative humidity ranging from about 70% to about 80%.

The present inventors observed that certain non-limiting embodiments of oral solid dosage forms according to the present disclosure retained at least 95% of an initial amount of levothyroxine in the oral solid dosage forms after storage for three months, four months, five months, six months, nine months, twelve months, eighteen months, or twenty-four months under standard testing conditions including a temperature of about 23° C. to about 27° C. and a relative humidity ranging from about 55% to about 65%. The present inventors observed that certain non-limiting embodiments of oral solid dosage forms according to the present disclosure retained at least 95% of an initial amount of levothyroxine after storage for three months or six months under intermediate testing conditions including a temperature of about 28° C. to about 32° C. and a relative humidity ranging from about 60% to about 70%. The present inventors also observed that certain embodiments of oral solid dosage forms according to the present disclosure retained at least 95% of an initial amount of levothyroxine after storage for one month, two months, three months, four months, five months, or six months under accelerated testing conditions including a temperature of about 38° C. to about 42° C. and a relative humidity ranging from about 70% to about 80%. The present inventors also observed that certain embodiments of oral solid dosage forms according to the present disclosure disposed in a sealed container retained at least 95% of an initial amount of levothyroxine for at least fifteen days when stored at a temperature of about 58° C. to about 62° C.

The present inventors observed that certain non-limiting embodiments of oral solid dosage forms according to the present disclosure retained at least 90% of an initial amount of liothyronine after storage for three months, four months, five months, six months, nine months, twelve months, eighteen months, or twenty-four months under standard testing conditions including a temperature of about 23° C. to about 27° C. and a relative humidity ranging from about 55% to about 65%. The present inventors observed that certain non-limiting embodiments of oral solid dosage forms according to the present disclosure retained at least 90% of an initial amount of liothyronine after storage for three months or six months under intermediate testing conditions including a temperature of about 28° C. to about 32° C. and a relative humidity ranging from about 60% to about 70%. The present inventors also observed that certain embodiments of oral solid dosage forms according to the present disclosure may retain at least 90% of an initial amount of liothyronine after storage for one month, two months, three months, four months, five months, or six months under accelerated testing conditions including a temperature of about 38° C. to about 42° C. and a relative humidity ranging from about 70% to about 80%.

The present inventors observed that certain non-limiting embodiments of oral solid dosage forms according to the present disclosure retained at least 92% of an initial amount of liothyronine after storage for three months, four months, five months, six months, nine months, twelve months, eighteen months, or twenty-four months under standard testing conditions including a temperature of about 23° C. to about 27° C. and a relative humidity ranging from about 55% to about 65%. The present inventors observed that certain non-limiting embodiments of oral solid dosage forms according to the present disclosure retained at least 92% of an initial amount of liothyronine after storage for three months or six months under intermediate testing conditions including a temperature of about 28° C. to about 32° C. and a relative humidity ranging from about 60% to about 70%. The present inventors also observed that certain embodiments of oral solid dosage forms according to the present disclosure retained at least 92% of an initial amount of liothyronine after storage for one month, two months, three months, four months, five months, or six months under accelerated testing conditions including a temperature of about 38° C. to about 42° C. and a relative humidity ranging from about 70% to about 80%.

The present inventors observed that certain non-limiting embodiments of oral solid dosage forms according to the present disclosure retained at least 95% of an initial amount of liothyronine after storage for three months, four months, five months, six months, nine months, twelve months, eighteen months, or twenty-four months under standard testing conditions including a temperature of about 23° C. to about 27° C. and a relative humidity ranging from about 55% to about 65%. The present inventors observed that certain non-limiting embodiments of oral solid dosage forms according to the present disclosure retained at least 95% of an initial amount of liothyronine after storage for three months or six months under intermediate testing conditions including a temperature of about 28° C. to about 32° C. and a relative humidity ranging from about 60% to about 70%. The present inventors also observed that certain embodiments of oral solid dosage forms according to the present disclosure retained at least 95% of an initial amount of liothyronine after storage for one month, two months, three months, four months, five months, or six months under accelerated testing conditions including a temperature of about 38° C. to about 42° C. and a relative humidity ranging from about 70% to about 80%. The present inventors also observed that certain embodiments of oral solid dosage forms according to the present disclosure disposed in a sealed container retained at least 95% of an initial amount of liothyronine for at least fifteen days when the containers were stored at a temperature of about 58° C. to about 62° C.

Following are non-limiting examples of oral dosage forms and methods of making oral dosage forms according to the present disclosure.

### EXAMPLES

### Example 1

This example describes a non-limiting embodiment of a dry layering/mixing and tableting process that may be used to make embodiments of oral solid dosage form according to the present disclosure.

A quarter of a total required amount of a powdered diluent (for example, AVICEL® HFE-102 powder or AVICEL® PH 102 powder) was screened through a #30 mesh screen, and the screened material was mixed in a V-blender for 1 minute at 25 RPM. The screened and mixed portion of the diluent powder formed a first powder layer in the V-blender. Next, a screened blend was prepared by adding the remainder portion of the powdered diluent to a bag of an amount of powdered glidant (for example, colloidal silicon dioxide) and screening the resulting blend of powders through a #30 mesh screen into a high-density polyethylene (HDPE) bag. An amount of powdered disintegrant (for example, sodium starch glycolate) was then screened through a #30 mesh screen into the bag. The screened blend of the diluent powder remainder portion, the glidant powder, and the disintegrant powder from the HDPE bag was then mixed in the V-blender at 25 RPM. The screened and mixed mixture of the diluent powder, glidant powder, and disintegrant powder formed a second layer in the V-blender on top of the first layer. Next, an amount of porcine thyroid powder was slowly mixed in the V-blender for 8 minutes at 25 RPM and disposed on the second layer in the V-blender to form a third layer. Next, an amount of lubricant (for example, calcium stearate) was screened through a #30 mesh and mixed in the V-blender for 5 minutes at 25 RPM. The screened and mixed portion of the lubricant material was disposed on the third layer in the V-blender and formed a fourth layer. The layered arrangement was then mixed in the V-blender until uniformly combined to form a final powder blend.

Multiple small quantities of the final powder blend were removed from the V-blender using a cradle discharge method over short time periods to prevent segregation of the ingredients. Measured portions of the final powder blend removed from the V-blender were compressed to a hardness of 1 to 12 Kp using a PROTAB 300 tableting press (ACG) into 32.5 mg, 65 mg, 130 mg, 260 mg, 390 mg, 520 mg, and 650 mg tablets. The tablets were packaged in HDPE containers (i.e., bottles) having an internal volume of either about 30 cm³ or about 75 cm³.

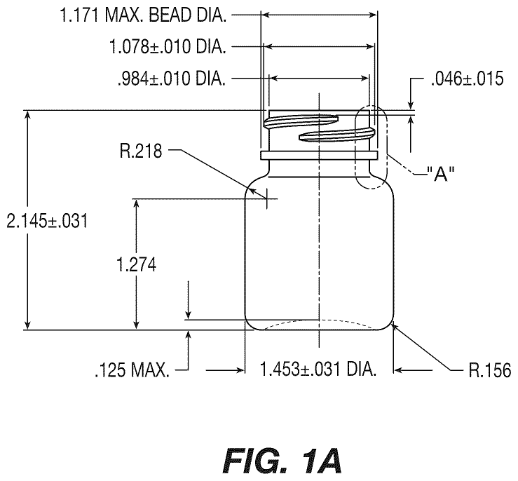

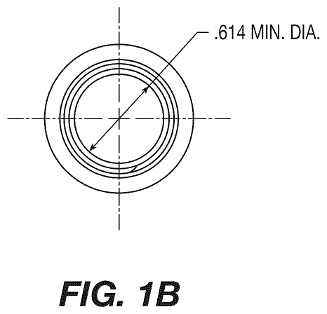

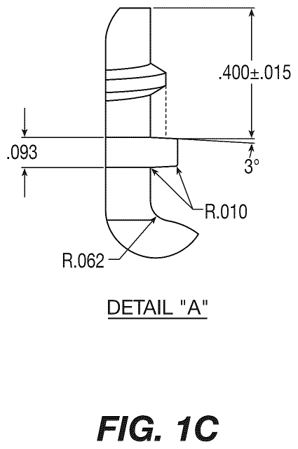

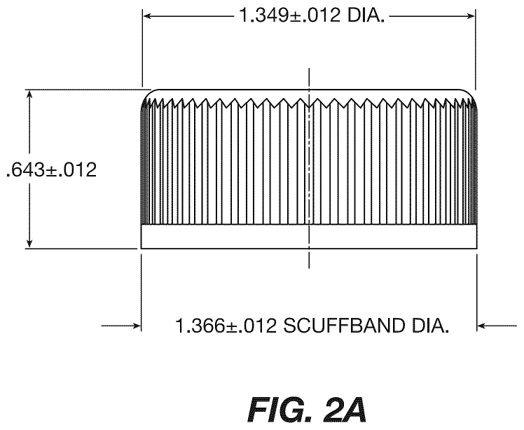

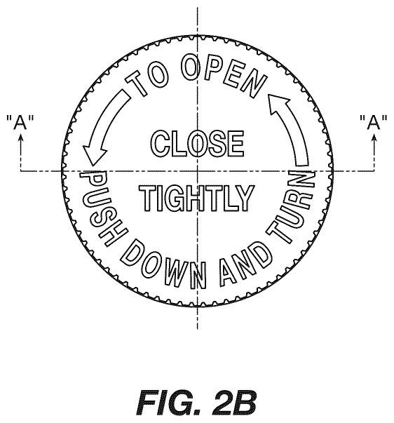

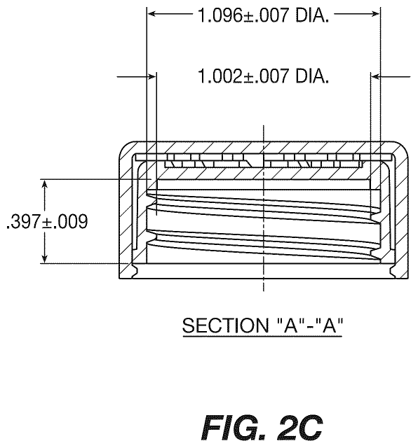

About 10 to about 20 of the tablets and 0, 1, or 2 silica gel desiccant canisters (commercially available from, e.g., Clariant International, Colton, CA) (each canister including about 2 g of desiccant) were disposed in the 30 cm³ HDPE containers (shown in FIGS. 1A, 1B, and 1C). In certain of the 30 cm³ containers including about 10 to 20 tablets, 0, 1, or 2 PHARMAKEEP® oxygen absorber canisters (commercially available from Clariant International) (each canister including about 2 g of oxygen absorber) also were disposed. The 30 cm³ containers were sealed with a sealing closure (depicted in FIGS. 2A, 2B, and 2C), providing a packaged immediate release oral solid dosage form product.

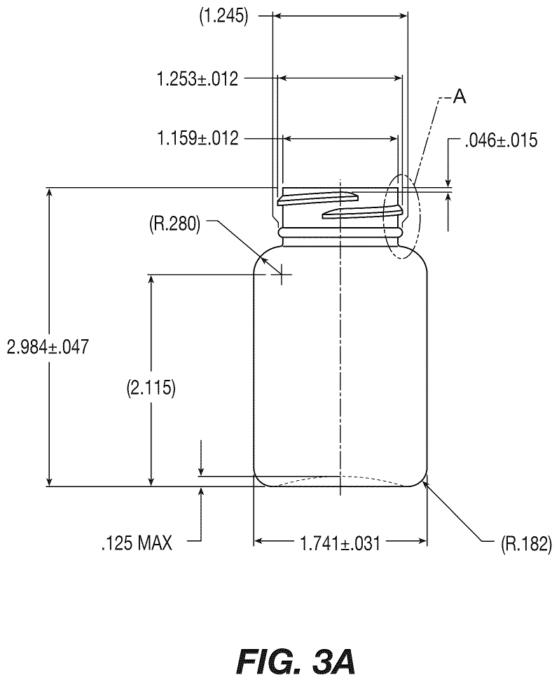

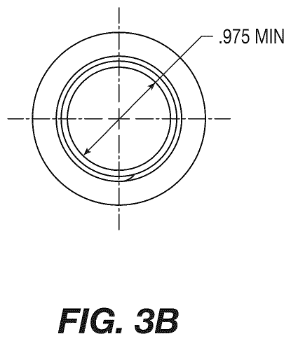

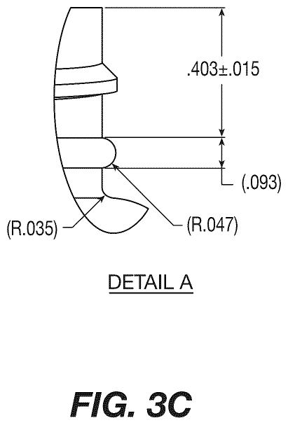

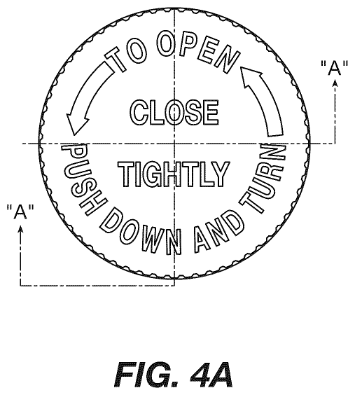

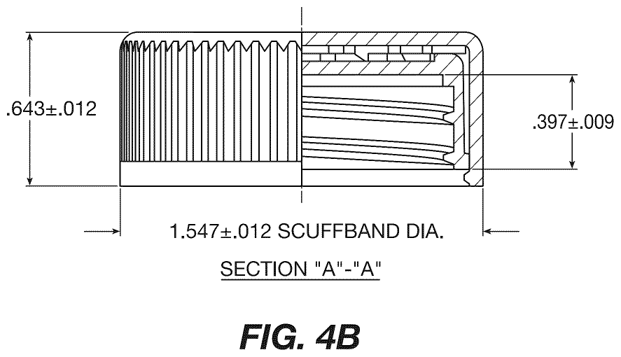

About 90 to about 100 the tablets and 0, 1, or 2 of the silica gel desiccant canisters were disposed in the 75 cm³ containers (shown in FIGS. 3A, 3B, and 3C). In certain of the 75 cm³ containers including about 90 to 100 tablets, 0, 1, or 2 of the PHARMAKEEP® oxygen absorber canisters also were disposed. The 75 cm³ containers were sealed with a sealing closure (shown in FIGS. 4A and 4B), providing a packaged immediate release oral solid dosage form product.

### Example 2

Tablets individually including 16.25 mg, 32.5 mg, 65.0 mg, 97.5 mg, 130 mg, 195 mg, 260 mg, and 325 mg of porcine thyroid powder were prepared by compressing portions of several uniform mixtures made using the method generally described in Example 1. Each of the tablets included porcine thyroid powder, microcrystalline cellulose (MCC), sodium starch glycolate, colloidal silicon dioxide, and calcium stearate. Table 1 below provides the mass and weight/weight concentration of the ingredients in individual tablets comprising 16.25 mg, 32.5 mg, 65.0 mg, 97.5 mg, 130 mg, 195 mg, 260 mg, and 325 mg porcine thyroid powder per tablet, which were referred to as the “strength” of the individual tablets. All tablet strengths listed in Table 1 included the same ingredients in the same weight percentage concentrations.

TABLE 1
Composition of tablets

| | All Tablet | 16.25 | 32.5 | 65.0 | 97.5 | 130.0 | 195.0 | 260.0 | 325 |
|---|---|---|---|---|---|---|---|---|---|
| | Strengths | mg | mg | mg | mg | mg | mg | mg | mg |
| Ingredient | % w/w | mg/ | mg/ | mg/ | mg/ | mg/ | mg/ | mg/ | mg/ |
| | | tablet | tablet | tablet | tablet | tablet | tablet | tablet | tablet |
| Porcine | 50.00 | 16.25 | 32.5 | 65.0 | 97.5 | 130.0 | 195.0 | 260.0 | 325.00 |
| thyroid | | | | | | | | | |
| powder, | | | | | | | | | |
| USP | | | | | | | | | |
| Avicel ® HFE | 44.15 | 14.35 | 28.7 | 57.4 | 86.09 | 114.79 | 172.10 | 229.58 | 286.98 |
| 102 Powder | | | | | | | | | |
| Sodium | 2.54 | 0.83 | 1.65 | 3.3 | 4.95 | 6.60 | 9.91 | 13.21 | 16.51 |
| Starch | | | | | | | | | |
| Glycolate | | | | | | | | | |
| (SSG) | | | | | | | | | |
| Colloidal | 2.50 | 0.81 | 1.63 | 3.25 | 4.88 | 6.50 | 9.75 | 13.00 | 16.25 |
| Silicon | | | | | | | | | |
| Dioxide | | | | | | | | | |
| Calcium | 0.81 | 0.26 | 0.53 | 1.05 | 1.58 | 2.11 | 3.16 | 4.21 | 5.27 |
| Stearate | | | | | | | | | |
| Total | 100.00 | 32.5 | 65.0 | 130.0 | 195.0 | 260.0 | 390.00 | 520.00 | 650.00 |

Given the differing amounts of porcine thyroid powder included in the various tablet strengths, the tablets of this Example 2 included an amount of levothyroxine in a range of about 9.5 μg up to about 190 μg and an amount of liothyronine in a range of about 2.2 μg up to about 45 μg. More specifically, the individual tablet strengths included the per tablet levothyroxine and liothyronine amounts listed in Table 2.

TABLE 2
Levothyroxine and liothyronine amounts

| | 16.25 | 32.5 | 65.0 | 97.5 | 130.0 | 195.0 | 260.0 | |
|---|---|---|---|---|---|---|---|---|
| Strength | mg | mg | mg | mg | mg | mg | mg | 325.0 mg |
| Levothyroxine | 9.5 μg | 19 μg | 38 μg | 57 μg | 76 μg | 114 μg | 152 μg | 190 μg |
| Liothyronine | 2.2 μg | 4.5 μg | 9 μg | 13.5 μg | 18 μg | 27 μg | 36 μg | 45 μg |

### Example 3

A study was conducted to assess the stability of embodiments of oral solid dosage forms according to the present disclosure.

Tablets including 16.25 mg of porcine thyroid powder were prepared by compressing portions of several uniform mixtures made using the method generally described in Example 1. Each of the mixtures included porcine thyroid powder, microcrystalline cellulose (MCC), sodium starch glycolate, colloidal silicon dioxide, and calcium stearate. The MCC was included as a component of Avicel® HFE 102 powder. Mannitol was also a component of the Avicel® HFE 102 powder. Table 3 below provides the mass and weight/weight concentration of the ingredients in individual tablets comprising 16.25 mg porcine thyroid powder per tablet produced in Batch NB539-111A.

TABLE 3
Composition of tablets in Batch NB539-111A

| Ingredient | % w/w | mg/unit |
|---|---|---|
| Porcine thyroid powder, USP | 50.00 | 16.25 |
| Avicel ® HFE 102 powder | 44.15 | 14.35 |
| Sodium Starch Glycolate (SSG) | 2.54 | 0.83 |
| Colloidal Silicon Dioxide | 2.50 | 0.81 |
| Calcium Stearate | 0.81 | 0.26 |
| Total | 100.00 | 32.5 |

The 16.25 mg of porcine thyroid powder in each tablet comprised about 9.5 μg of levothyroxine and about 2.2 μg of liothyronine. Solid dosage forms including different amounts of the active agents may be formed in a dose similar manner by adjusting the amount of the active ingredient, along with the amount of inactive ingredients to keep the mass of the dosage forms the same.

For purposes of conducting the stability study, the tablets of Batch NB539-111A were disposed in container closure systems including a container comprising an internal volume of either about 30 cm³ (including 10 to 20 tablets) or about 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed. As an alternative, air may be displaced within a container by disposing an inert pill packing material, for example, rayon or cotton, in the container after tablet loading and before induction sealing.

Three studies were conducted to assess the stability of the levothyroxine and liothyronine in the tablets produced in Batch NB539-111A. The stability studies involved the following testing periods and conditions:
Storing under standard testing conditions (25° C.±2° C. and 60%±5% relative humidity) for up to five months.
Storing under accelerated testing conditions (40° C.±2° C. and 75%±5% relative humidity) for up to five months.
Storing under extreme testing conditions (60° C.±2° C.) for 7 or 15 days.

A standard testing conditions stability study of the tablets of Batch NB539-111A contained in the container closure system was conducted over a period of five months to determine the rate of physical or chemical degradation of the levothyroxine and liothyronine included in tablets when stored under the standard environmental conditions.

Container closure systems prepared as described above containing tablets including about 9.5 μg of levothyroxine and about 2.2 μg of liothyronine per tablet were placed upright in a calibrated environmental chamber and maintained in an upright position during the stability study. The sealed containers including either 10 to 20 or 90 to 100 tablets were maintained at 25° C.±2° C. and 60%±5% relative humidity, uninterrupted (except for the addition or withdrawal of test samples), for a period of 15 days, one month, two months, or five months. Tablets were removed from the storage conditions at the specified time points and tested for levothyroxine and liothyronine content using high pressure liquid chromatography.

Considering levothyroxine content, shelf life of tablets of Batch NB539-111A was estimated to be at least five months when stored under standard testing conditions. Table 4 provides the five-month stability assay results for tablets of Batch NB539-111A. The stability assay showed that greater than 95 percent of the original levothyroxine content was retained in tablets stored under standard testing conditions for five months.

Considering liothyronine content, shelf life of tablets of Batch NB539-111A was estimated to be at least five months when stored under standard testing conditions. As shown in Table 4, the stability assay showed that greater than 95 percent of the original liothyronine content was retained in tablets stored under standard testing conditions for five months.

Accelerated condition stability samples were placed upright in an environmental chamber and maintained in an upright position during the stability testing. As discussed, each tablet was disposed in the sealed container containing either 10 to 20 or 90 to 100 tablets from Batch NB539-111A, and each tablet included about 9.5 μg of levothyroxine and about 2.2 μg of liothyronine per tablet. The sealed containers were maintained at 40° C.±2° C. and 75%±5% relative humidity, uninterrupted, (except for the addition or withdrawal of test samples) for a period of 15 days, one month, two months, or five months. Tablets were removed from the storage conditions at the specified time points and tested for levothyroxine and liothyronine content using high pressure liquid chromatography.

Considering levothyroxine content, shelf life of tablets of Batch NB539-111A was estimated to be at least five months when stored under accelerated testing conditions. Table 4 provides the five-month stability assay results for tablets of Batch NB539-111A stored under accelerated testing conditions. The stability assay showed that greater than 95 percent of the original levothyroxine content was retained in tablets stored under accelerated testing conditions for five months.

Considering liothyronine content, shelf life of tablets of Batch NB539-111A was estimated to be at least five months when stored under accelerated testing conditions. As shown in Table 4, the stability assay showed that greater than 95 percent of the original liothyronine content was retained in tablets stored under accelerated testing conditions for five months.

An extreme testing conditions stability study was conducted in which tablets of Batch NB539-111A were stored under extreme testing conditions for a period of 7 or 15 days to determine the rate of physical or chemical degradation of the original content of about 9.5 μg of levothyroxine and about 2.2 μg of liothyronine in the tablets. All extreme testing conditions stability samples (each sample comprising a container closure system containing either 10 to 20 or 90 to 100 tablets of Batch NB539-111A) were placed upright in an environmental chamber and maintained in an upright position during the stability study. All of the containers were maintained at 60° C.±2° C., uninterrupted (except for the addition or withdrawal of test samples), for a period of 7 or 15 days. Tablets were removed from the storage conditions at the specified time points and tested for levothyroxine and liothyronine content using high pressure liquid chromatography.

Considering levothyroxine content, shelf life of tablets of Batch NB539-111A was estimated to be at least fifteen days when stored under extreme testing conditions. Table 4 provides the fifteen-day stability assay results for tablets of Batch NB539-111A. The stability assay showed that greater than 95 percent of the original levothyroxine content was retained in tablets stored under extreme testing conditions for fifteen days.

Considering liothyronine content, shelf life of tablets of Batch NB539-111A was estimated to be at least fifteen days when stored under extreme testing conditions. As shown in Table 4, the stability assay showed that greater than 95 percent of the original liothyronine content was retained in tablets stored under extreme testing conditions for fifteen days.

Table 4 reports stability data for the oral solid dosage form samples (Batch NB539-111A) tested under standard, accelerated, and extreme conditions. Table 4 identifies the bottle size for storing each test sample and the number of silica gel desiccant canisters and PHARMAKEEP® oxygen absorber canisters included in the sealed container. The assay percentages listed in Table 4 are based on expected (“label claim”) contents of 9.5 μg of levothyroxine and 2.2 μg of liothyronine per tablet based on the 16.25 mg of powdered porcine thyroid included in each tablet of Batch NB539-111A. Table 4 includes both uncorrected assay percentages and assay percentages corrected to factor in the actual mass of the tablets evaluated.

TABLE 4
Stability assay results for tablets of Batch NB539-111A

| | | Silica | Oxygen | | | | |
|---|---|---|---|---|---|---|---|
| Time and | Bottle | Gel | Absorber- | Assay % | | Assay % (Wt Corrected) | |
| Conditions | Size | Desiccant | PHARMAKEEP | Liothyronine | Levothyroxine | Liothyronine | Levothyroxine |
| Time Zero | NA | NA | NA | 104.1 | 107.8 | 96.1 | 99.6 |
| 7 Days, | 75 cm³ | 0 | 0 | 101.7 | 105.0 | 93.9 | 97.0 |
| Extreme | | 1 | 1 | 104.6 | 108.7 | 96.6 | 100.4 |
| Testing | | 2 | 1 | 104.6 | 108.7 | 96.6 | 100.4 |
| Conditions | | 1 | 2 | 104.5 | 108.6 | 96.5 | 100.3 |
| | | 2 | 2 | 103.7 | 107.3 | 95.8 | 99.1 |
| | 30 cm³ | 0 | 0 | 101.8 | 105.0 | 94.0 | 97.0 |
| | | 1 | 1 | 103.7 | 107.5 | 95.8 | 99.3 |
| 15 Days, | 75 cm³ | 0 | 0 | 101.7 | 104.7 | 93.9 | 96.7 |
| Extreme | | 1 | 1 | 103.2 | 106.5 | 95.3 | 98.3 |
| Testing | | 2 | 1 | 105.1 | 108.8 | 97.1 | 100.5 |
| Conditions | | 1 | 2 | 104.5 | 108.1 | 96.5 | 99.8 |
| | | 2 | 2 | 104.1 | 107.6 | 96.1 | 99.4 |
| | 30 cm³ | 0 | 0 | 102.3 | 105.4 | 94.5 | 97.3 |
| | | 1 | 1 | 103.2 | 106.6 | 95.3 | 98.4 |
| 15 Days, | 75 cm³ | 0 | 0 | 103.5 | 107.4 | 95.6 | 99.2 |
| Standard | | 1 | 1 | 102.5 | 105.8 | 94.7 | 97.7 |
| Testing | | 2 | 1 | 102.5 | 106.1 | 94.7 | 98.0 |
| Conditions | | 1 | 2 | 102.9 | 106.5 | 95.0 | 98.3 |
| | | 2 | 2 | 103.3 | 106.5 | 95.4 | 98.3 |
| | 30 cm³ | 0 | 0 | 104.5 | 108.1 | 96.5 | 99.8 |
| | | 1 | 1 | 102.5 | 106.0 | 94.7 | 97.9 |
| 15 Days, | 75 cm³ | 0 | 0 | 105.2 | 108,5 | 97.1 | 100.2 |
| Accelerated | | 1 | 1 | 106.4 | 110.0 | 98.2 | 101.6 |
| Testing | | 2 | 1 | 107.0 | 110.6 | 98.8 | 102.1 |
| Conditions | | 1 | 2 | 107.3 | 111.1 | 99.1 | 102.6 |
| | | 2 | 2 | 107.4 | 111.0 | 99.2 | 102.5 |
| | 30 cm³ | 0 | 0 | 105.4 | 108.6 | 97.3 | 100.2 |
| | | 1 | 1 | 107.0 | 110.4 | 98.8 | 101.9 |
| 1 Month, | 75 cm³ | 0 | 0 | 106.7 | 111.1 | 98.5 | 102.6 |
| Standard | | 1 | 1 | 105.1 | 108.5 | 97.1 | 100.2 |
| Testing | | 2 | 1 | 106.8 | 111.3 | 98.6 | 102.7 |
| Conditions | | 1 | 2 | 107.3 | 111.2 | 99.0 | 102.7 |
| | | 2 | 2 | 104.7 | 109.2 | 96.7 | 100.8 |
| | 30 cm³ | 0 | 0 | 106.6 | 111.0 | 98.5 | 102.5 |
| | | 1 | 1 | 105.9 | 110.3 | 97.8 | 101.9 |
| 1 Month, | 75 cm³ | 0 | 0 | 103.7 | 105.7 | 95.8 | 97.6 |
| Accelerated | | 1 | 1 | 105.5 | 108.5 | 97.4 | 100.2 |
| Testing | | 2 | 1 | 106.4 | 111.2 | 98.3 | 102.6 |
| Conditions | | 1 | 2 | 107.5 | 110.8 | 99.2 | 102.3 |
| | | 2 | 2 | 103.6 | 107.2 | 95.7 | 99.0 |
| | 30 cm³ | 0 | 0 | 105,8 | 109.6 | 97.7 | 101.2 |
| | | 1 | 1 | 105.8 | 109.6 | 97.7 | 101.2 |
| 2 Months, | 75 cm³ | 1 | 1 | 102.8 | 107.9 | 94.9 | 99.6 |
| Accelerated | | | | | | | |
| Testing | | | | | | | |
| Conditions | | | | | | | |
| 2 Months, | | 1 | 1 | 103.2 | 108.4 | 95.3 | 100.1 |
| Standard | | | | | | | |
| Testing | | | | | | | |
| Conditions | | | | | | | |
| 2 Months, | 30 cm³ | 1 | 1 | 102.6 | 108.2 | 94.7 | 99.9 |
| Accelerated | | | | | | | |
| Testing | | | | | | | |
| Conditions | | | | | | | |
| 2 Months, | | 1 | 1 | 102.9 | 107.8 | 95.0 | 99.6 |
| Standard | | | | | | | |
| Testing | | | | | | | |
| Conditions | | | | | | | |
| 5 Months, | 75 cm³ | 1 | 1 | 102.1 | 107.0 | 94.3 | 98.8 |
| Accelerated | | | | | | | |
| Testing | | | | | | | |
| Conditions | | | | | | | |
| 5 Months, | | 1 | 1 | 102.6 | 107.1 | 94.7 | 98.9 |
| Standard | | | | | | | |
| Testing | | | | | | | |
| Conditions | | | | | | | |
| 5 Months, | 30 cm³ | 1 | 1 | 101.6 | 106.4 | 93.8 | 98.3 |
| Accelerated | | | | | | | |
| Testing | | | | | | | |
| Conditions | | | | | | | |
| 5 Months, | | 1 | 1 | 102.5 | 107.5 | 94.7 | 99.3 |
| Standard | | | | | | | |
| Testing | | | | | | | |
| Conditions | | | | | | | |

With reference to the results shown in Table 4, the present inventors surprisingly observed that oral solid dosage forms produced in Batch NB539-111A retained about 99% of the original content of levothyroxine when stored under standard testing conditions at 25° C.±2° C. and 60%±5% relative humidity for a period of five months. Even when tablets were stored under accelerated conditions (40° C.±2° C. and 75%±5% relative humidity) for five months, tablets produced in Batch NB539-111A retained over 98% of the original levothyroxine content (a decrease in the levothyroxine content of the tablets of less than two percent).

With reference to the results shown in Table 4, the present inventors also surprisingly observed that oral solid dosage forms produced in Batch NB539-111A retained about 95% of the original content of liothyronine when stored under standard testing conditions at 25° C.±2° C. and 60%±5% relative humidity for a period of five months. Even when tablets were stored under accelerated conditions (40° C.±2° C. and 75%±5% relative humidity) for five months, tablets produced in Batch NB539-111A retained over 94% of the original liothyronine content.

### Example 4

An additional study was conducted to assess the stability of oral solid dosage forms according to the present disclosure.

Tablets including 130 mg of porcine thyroid powder were prepared by compressing portions of several uniform mixtures made using the method generally described in Example 1. Each of the mixtures included porcine thyroid powder, microcrystalline cellulose (MCC), sodium starch glycolate, colloidal silicon dioxide, and calcium stearate. The MCC was included as a component of Avicel® HFE 102 powder. Mannitol was also a component of the Avicel® HFE 102 powder. Table 5 below provides the mass and weight/weight concentration of the ingredients in individual tablets comprising 130 mg porcine thyroid powder per tablet produced in Batch NB539-111B.

TABLE 5
Composition of tablets in Batch NB539-111B

| Ingredients | % w/w | mg/unit |
|---|---|---|
| Porcine thyroid powder, USP | 50.00 | 130.0 |
| Avicel ® HFE 102 powder | 44.15 | 114.79 |
| Sodium Starch Glycolate | 2.54 | 6.6 |
| Colloidal Silicon Dioxide | 2.50 | 6.5 |
| Calcium Stearate | 0.81 | 2.11 |
| Total | 100.00 | 260.0 |

The 130 mg of porcine thyroid powder in each tablet comprised about 76 μg of levothyroxine and about 18 μg of liothyronine. Solid dosage forms including different amounts of the active agent may be formed in a dose similar manner by adjusting the amount of the active ingredient, along with the amount of inactive ingredients to keep the mass of the dosage forms the same.

For purposes of conducting the stability study, the tablets of Batch NB539-111B were disposed in container closure systems including a container comprising an internal volume of either 30 cm³ (including 10 to 20 tablets) or 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed. The sealed containers were stored in a calibrated environmental chamber under one of the standard, accelerated, and extreme testing conditions described in Example 3 for a predetermined time.

A standard testing conditions stability study of the tablets of Batch NB539-111B contained in the container closure system was conducted over a period of five months to determine the rate of physical or chemical degradation of the levothyroxine and liothyronine included in the tablets when stored under the standard conditions.

Container closure systems prepared as described above containing tablets including about 76 μg of levothyroxine and about 18 μg of liothyronine per tablet were placed upright in a calibrated environmental chamber and maintained in an upright position during the stability study. The sealed containers including either 10 to 20 or 90 to 100 tablets were maintained at 25° C.±2° C. and 60%±5% relative humidity, uninterrupted (except for the addition or withdrawal of test samples), for a period of 15 days, one month, two months, or five months. Tablets were removed from the storage conditions at the specified time points and tested for levothyroxine and liothyronine content using high pressure liquid chromatography.

Considering levothyroxine content, shelf life of tablets of Batch NB539-111B was estimated to be at least five months when stored under standard testing conditions. Table 6 provides the five-month stability assay results for tablets of Batch NB539-111B. The stability assay showed that greater than 95 percent of the original levothyroxine content was retained in tablets stored under standard testing conditions for five months.

Considering liothyronine content, shelf life of tablets of Batch NB539-111B was estimated to be at least five months when stored under standard testing conditions. As shown in Table 6, the stability assay showed that greater than 95 percent of the original liothyronine content was retained in tablets stored under standard testing conditions for five months.

Accelerated condition stability samples were placed upright in an environmental chamber and maintained in an upright position during the stability testing. As discussed, each tablet was disposed in the sealed container containing either 10 to 20 or 90 to 100 tablets from Batch NB539-111B, and each tablet included about 76 μg of levothyroxine and about 18 μg of liothyronine per tablet. The sealed containers were maintained at 40° C.±2° C. and 75% #5% relative humidity, uninterrupted, (except for the addition or withdrawal of test samples) for a period of 15 days, one month, two months, or five months. Tablets were removed from the storage conditions at the specified time points and tested for levothyroxine and liothyronine content using high pressure liquid chromatography.

Considering levothyroxine content, shelf life of tablets of Batch NB539-111B was estimated to be at least five months when stored under accelerated testing conditions. Table 6 provides the five-month stability assay results for tablets of Batch NB539-111B stored under accelerated testing conditions. The stability assay showed that greater than 95 percent of the original levothyroxine content was retained in tablets stored under accelerated testing conditions for five months.

Considering liothyronine content, shelf life of tablets of Batch NB539-111B was estimated to be at least five months when stored under accelerated testing conditions. As shown in Table 6, the stability assay showed that greater than 95 percent of the original liothyronine content was retained in tablets stored under accelerated testing conditions for five months.

An extreme testing conditions stability study was conducted in which tablets of Batch NB539-111B were stored under extreme testing conditions for a period of 7 or 15 days to determine the rate of physical or chemical degradation of the original 76 μg of levothyroxine and 18 μg of liothyronine in the tablets. All extreme testing conditions stability samples (each sample comprising a container closure system containing either 10 to 20 or 90 to 100 tablets of Batch NB539-111B) were placed upright in an environmental chamber and maintained in an upright position during the stability study. All of the containers were maintained at 60° C.±2° C., uninterrupted (except for the addition or withdrawal of test samples), for a period of 7 or 15 days. Samples were removed from conditions at the specified time points and tested for levothyroxine and liothyronine content. Tablets were removed from the storage conditions at the specified time points and tested for levothyroxine and liothyronine content using high pressure liquid chromatography.

Considering levothyroxine content, shelf life of tablets of Batch NB539-111B was estimated to be at least fifteen days when stored under extreme testing conditions. Table 6 provides the fifteen-day stability assay results for tablets of Batch NB539-111B. The stability assay showed that greater than 95 percent of the original levothyroxine content was retained in tablets stored under extreme testing conditions for fifteen days.

Considering liothyronine content, shelf life of tablets of Batch NB539-111B was estimated to be at least fifteen days when stored under extreme testing conditions. As shown in Table 6, the stability assay showed that greater than 95 percent of the original liothyronine content was retained in tablets stored under extreme testing conditions for fifteen days.

Table 6 reports stability data for the oral solid dosage form samples (Batch NB539-111B) tested under standard, accelerated, and extreme conditions. Table 6 identifies the bottle size for storing each test sample and the number of silica gel desiccant canisters and PHARMAKEEP® oxygen absorber canisters included in the sealed container. The assay percentages listed in Table 6 are based on expected contents of 76 μg of levothyroxine and 18 μg of liothyronine per tablet based on the 130 mg of powdered porcine thyroid included in each tablet from Batch NB539-111B. Table 6 includes both uncorrected assay percentages and assay percentages corrected to factor in the actual mass of the tablets evaluated.

TABLE 6
Stability results for tablets of Batch NB539-111B

| | | | Oxygen | | | | |
|---|---|---|---|---|---|---|---|
| Time and | Bottle | Silica Gel | Absorber- | Assay % | | Assay % (Wt Corrected) | |
| Conditions | Size | Desiccant | PHARMAKEEP | Liothyronine | Levothyroxine | Liothyronine | Levothyroxine |
| Time Zero | NA | NA | NA | 104.8 | 108.1 | 96.8 | 99.8 |
| 7 Days, Extreme | 75 cm³ | 0 | 0 | 103.4 | 106.7 | 95.5 | 98.5 |
| Testing Conditions | 75 cm³ | 1 | 1 | 103.3 | 107.7 | 95.4 | 99.5 |
| | 75 cm³ | 2 | 1 | 104.1 | 108.3 | 96.1 | 100.0 |
| | 75 cm³ | 1 | 2 | 104.0 | 108.0 | 96.0 | 99.7 |
| | 75 cm³ | 2 | 2 | 103.7 | 107.7 | 95.8 | 99.5 |
| | 30 cm³ | 0 | 0 | 103.1 | 106.4 | 95.2 | 98.3 |
| | 30 cm³ | 1 | 1 | 103.2 | 107.1 | 95.3 | 98.9 |
| 15 Days, Extreme | 75 cm³ | 0 | 0 | 103.5 | 106.3 | 95.6 | 98.2 |
| Testing Conditions | 75 cm³ | 1 | 1 | 104.0 | 107.7 | 96.0 | 99.5 |
| | 75 cm³ | 2 | 1 | 104.2 | 108.0 | 96.2 | 99.7 |
| | 75 cm³ | 1 | 2 | 104.3 | 108.2 | 96.3 | 99.9 |
| | 75 cm³ | 2 | 2 | 104.5 | 108.0 | 96.5 | 99.7 |
| | 30 cm³ | 0 | 0 | 103.5 | 106.3 | 95.6 | 98.2 |
| | 30 cm³ | 1 | 1 | 104.3 | 107.7 | 96.3 | 99.5 |
| 15 Days, Standard | 75 cm³ | 0 | 0 | 106.3 | 110.0 | 98.2 | 101.6 |
| Testing Conditions | 75 cm³ | 1 | 1 | 105.7 | 109.2 | 97.6 | 100.8 |
| | 75 cm³ | 2 | 1 | 106.2 | 109.9 | 98.1 | 101.5 |
| | 75 cm³ | 1 | 2 | 106.5 | 110.4 | 98.3 | 102.0 |
| | 75 cm³ | 2 | 2 | 105.8 | 109.4 | 97.7 | 101.0 |
| | 30 cm³ | 0 | 0 | 104.7 | 108.4 | 96.7 | 100.1 |
| | 30 cm³ | 1 | 1 | 106.0 | 109.7 | 97.9 | 101.3 |
| 15 Days, | 75 cm³ | 0 | 0 | 106.1 | 109.7 | 98.0 | 101.3 |
| Accelerated Testing | 75 cm³ | 1 | 1 | 107.1 | 110.7 | 98.9 | 102.2 |
| Conditions | 75 cm³ | 2 | 1 | 106.7 | 110.4 | 98.5 | 101.9 |
| | 75 cm³ | 1 | 2 | 106.8 | 110.6 | 98.7 | 102.2 |
| | 75 cm³ | 2 | 2 | 106.9 | 110.5 | 98.8 | 102.1 |
| | 30 cm³ | 0 | 0 | 107.4 | 110.8 | 99.1 | 102.3 |
| | 30 cm³ | 1 | 1 | 106.3 | 110.0 | 98.2 | 101.6 |
| 1M, Standard | 75 cm³ | 0 | 0 | 108.2 | 113.2 | 99.9 | 104.5 |
| Testing Conditions | 75 cm³ | 1 | 1 | 108.4 | 113.6 | 100.1 | 104.9 |
| | 75 cm³ | 2 | 1 | 108.6 | 113.8 | 100.3 | 105.1 |
| | 75 cm³ | 1 | 2 | 108.5 | 113.2 | 100.2 | 104.5 |
| | 75 cm³ | 2 | 2 | 108.8 | 113.6 | 100.5 | 104.9 |
| | 30 cm³ | 0 | 0 | 108.0 | 112.5 | 99.8 | 103.9 |
| | 30 cm³ | 1 | 1 | 107.9 | 112.3 | 99.7 | 103.7 |
| 1M, Accelerated | 75 cm³ | 0 | 0 | 109.2 | 113.0 | 100.8 | 104.3 |
| Testing Conditions | 75 cm³ | 1 | 1 | 110.3 | 114.3 | 101.8 | 105.5 |
| | 75 cm³ | 2 | 1 | 110.3 | 114.3 | 101.9 | 105.5 |
| | 75 cm³ | 1 | 2 | 109.8 | 113.8 | 101.4 | 105.1 |
| | 75 cm³ | 2 | 2 | 110.3 | 114.1 | 101.9 | 105.4 |
| | 30 cm³ | 0 | 0 | 109.2 | 112.4 | 100.9 | 103.8 |
| | | 1 | 1 | 110.2 | 113.8 | 101.8 | 105.1 |
| 2M, Standard | 75 cm³ | 1 | 1 | 104.4 | 109.5 | 96.4 | 101.1 |
| Testing Conditions | | | | | | | |
| 2M, Accelerated | | 1 | 1 | 103.8 | 108.7 | 95.9 | 100.4 |
| Testing Conditions | | | | | | | |
| 2M, Standard | 30 cm³ | 1 | 1 | 104.7 | 109.6 | 96.7 | 101.2 |
| Testing Conditions | | | | | | | |
| 2M, Accelerated | | 1 | 1 | 103.7 | 108.7 | 95.8 | 100.4 |
| Testing Conditions | | | | | | | |
| 5M, Standard | 75 cm³ | 1 | 1 | 104.2 | 109.4 | 96.2 | 101.0 |
| Testing Conditions | | | | | | | |
| 5M, Accelerated | | 1 | 1 | 103.9 | 108.6 | 95.9 | 100.3 |
| Testing Conditions | | | | | | | |
| 5M, Standard | 30 cm³ | 1 | 1 | 104.7 | 109.7 | 96.7 | 101.3 |
| Testing Conditions | | | | | | | |
| 5M, Accelerated | | 1 | 1 | 103.4 | 108.1 | 95.5 | 99.8 |
| Testing Conditions | | | | | | | |

With reference to the results shown in Table 6, the inventors surprisingly observed that oral solid dosage forms produced in Batch NB539-111B retained 100% of the original content of levothyroxine when stored under standard testing conditions at 25° C.±2° C. and 60%±5% relative humidity for a period of five months. Even when tablets were stored under accelerated conditions (40° C.±2° C. and 75%±5% relative humidity) for five months, tablets produced in Batch NB539-111B retained about 100% of the original levothyroxine content.

With reference to the results shown in Table 6, the inventors also surprisingly observed that oral solid dosage forms produced in Batch NB539-111B retained more than 96% of the original content of liothyronine when stored under standard testing conditions at 25° C.±2° C. and 60%±5% relative humidity for a period of five months. Even when tablets were stored under accelerated conditions (40° C.±2° C. and 75%±5% relative humidity) for five months, tablets produced in Batch NB539-111B retained over 95% of the original liothyronine content.

### Example 5

A study was conducted to assess the stability of embodiments of oral solid dosage forms according to the present disclosure.

Tablets including 16.25 mg of porcine thyroid powder were prepared by compressing portions of several uniform mixtures made using the method generally described in Example 1. Each of the mixtures included porcine thyroid powder, microcrystalline cellulose (MCC), sodium starch glycolate, colloidal silicon dioxide, and calcium stearate. The MCC was included as a component of Avicel® HFE 102 powder. Mannitol was also a component of the Avicel® HFE 102 powder. Table 7 below provides the mass and weight/weight concentration of the ingredients in individual tablets comprising 16.25 mg porcine thyroid powder per tablet produced in Batches S45000121A, S45000121B, S45000221A, S45000221B, S45000321A, and S45000321B.

TABLE 7
Composition of tablets in Batches S45000121A, S45000121B,
S45000221A, S45000221B, S45000321A, and S45000321B

| Ingredient | % w/w | mg/unit |
|---|---|---|
| Porcine thyroid powder, USP | 50.00 | 16.25 |
| Avicel ® HFE 102 powder | 44.15 | 14.35 |
| Sodium Starch Glycolate (SSG) | 2.54 | 0.83 |
| Colloidal Silicon Dioxide | 2.50 | 0.81 |
| Calcium Stearate | 0.81 | 0.26 |
| Total | 100.00 | 32.5 |

The 16.25 mg of porcine thyroid powder in each tablet comprised about 9.5 μg of levothyroxine and about 2.2 μg of liothyronine. Solid dosage forms including different amounts of the active agents may be formed in a dose similar manner by adjusting the amount of the active ingredient, along with the amount of inactive ingredients to keep the mass of the dosage forms the same.

For purposes of conducting the stability study, the tablets of Batches S45000121A, S45000121B, S45000221A, S45000221B, S45000321A, and S45000321B were disposed in container closure systems including a container comprising an internal volume of either about 30 cm³ (including 10 to 20 tablets) or about 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed. As an alternative, air may be displaced within a container by disposing an inert pill packing material, for example, rayon or cotton, in the container after tablet loading and before induction sealing.

Three studies were conducted to assess the stability of the levothyroxine and liothyronine in the tablets produced in Batches S45000121A, S45000121B, S45000221A, S45000221B, S45000321A, and S45000321B. The stability studies involved the following testing periods and conditions:
Storing under standard testing conditions (25° C.±2° C. and 60%±5% relative humidity) for up to 24 months.
Storing under intermediate testing conditions (30° C.±2° C. and 65%±5% relative humidity) for up to 6 months.
Storing under accelerated testing conditions (40° C.±2° C. and 75%±5% relative humidity) for up to 6 months.

A standard testing conditions stability study of the tablets of Batches S45000121A, S45000121B, S45000221A, S45000221B, S45000321A, and S45000321B contained in the container closure system was conducted over a period of twenty-four months to determine the rate of physical or chemical degradation of the levothyroxine and liothyronine included in tablets when stored under the standard environmental conditions.

Container closure systems prepared as described above containing tablets including about 9.5 μg of levothyroxine and about 2.2 μg of liothyronine per tablet were placed upright in a calibrated environmental chamber and maintained in an upright position during the stability study. The sealed containers including either 10 to 20 or 90 to 100 tablets were maintained at 25° C.±2° C. and 60%±5% relative humidity, uninterrupted (except for the addition or withdrawal of test samples), for a period of three months, four months, five months, six months, nine months, twelve months, eighteen months, or twenty-four months. Tablets were removed from the storage conditions at the specified time points and tested for levothyroxine and liothyronine content using high pressure liquid chromatography.

Considering levothyroxine content, shelf life of tablets of Batches S45000121A, S45000121B, S45000221A, S45000221B, S45000321A, and S45000321B was estimated to be at least twenty-four months when stored under standard testing conditions. Table 8 provides the twenty-four month stability assay results for tablets of Batches S45000121A, S45000121B, S45000221A, S45000221B, S45000321A, and S45000321B. The stability assay showed that greater than 95 percent of the original levothyroxine content was retained in tablets stored under standard testing conditions for twenty-four months.

Considering liothyronine content, shelf life of tablets of Batches S45000121A, S45000121B, S45000221A, S45000221B, S45000321A, and S45000321B was estimated to be at least twenty-four months when stored under standard testing conditions. As shown in Table 8, the stability assay showed that greater than 95 percent of the original liothyronine content was retained in tablets stored under standard testing conditions for twenty-four months.

TABLE 8
Stability assay results under standard testing conditions for tablets of Batches S45000121A,
S45000121B, S45000221A, S45000221B, S45000321A, and S45000321B
Thyroid Tablets, USP 25° C./60% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 16.25 mg

| | | | | | | |
|---|---|---|---|---|---|---|
| S45000121A | 14 | 0 M | 97.0 | 100.4 | 05:01 | 3% |
| | | 3 M | 99.1 | 102.7 | 04:46 | 1% |
| | | 4 M | 98.7 | 100.9 | 04:59 | 1% |
| | | 5 M | 99.6 | 103.8 | 04:32 | 1% |
| | | 6 M | 96.0 | 99.2 | 04:42 | 1% |
| | | 9 M | 100.0 | 98.9 | 04:24 | 2% |
| | | 12 M | 101.1 | 101.5 | 04:18 | 1% |
| | | 18 M | 100.3 | 102.8 | 04:35 | 1% |
| | | 24 M | 100.9 | 105.6 | 04:31 | 2% |
| S45000121B | 90 | 0 M | 97.0 | 100.4 | 05:01 | 3% |
| | | 3 M | 98.9 | 102.6 | 05:03 | 1% |
| | | 4 M | 98.7 | 100.1 | 05:19 | 1% |
| | | 5 M | 98.3 | 98.7 | 05:03 | 2% |
| | | 6 M | 96.3 | 99.7 | 04:26 | 1% |
| | | 9 M | 97.4 | 99.8 | 04:48 | 2% |
| | | 12 M | 101.0 | 101.8 | 03:54 | 2% |
| | | 18 M | 99.6 | 100.4 | 04:26 | 2% |
| | | 24 M | 101.3 | 105.2 | 04:07 | 2% |
| S45000221A | 14 | 0 M | 98.6 | 103.2 | 04:34 | 3% |
| | | 3 M | 99.7 | 103.3 | 05:32 | 1% |
| | | 4 M | 98.9 | 101.3 | 05:30 | 1% |
| | | 5 M | 98.6 | 102.3 | 04:51 | 1% |
| | | 6 M | 97.2 | 100.2 | 05:23 | 1% |
| | | 9 M | 100.7 | 99.6 | 04:44 | 2% |
| | | 12 M | 102.1 | 102.3 | 03:30 | 1% |
| | | 18 M | 100.5 | 102.0 | 04:46 | 1% |
| | | 24 M | 102.0 | 107.0 | 03:44 | 2% |
| S45000221B | 90 | 0 M | 98.6 | 103.2 | 04:34 | 3% |
| | | 3 M | 99.6 | 101.2 | 05:06 | 1% |
| | | 4 M | 98.7 | 99.8 | 04:52 | 1% |
| | | 5 M | 98.3 | 99.2 | 04:50 | 1% |
| | | 6 M | 96.4 | 100.3 | 04:02 | 1% |
| | | 9 M | 99.1 | 98.7 | 04:40 | 2% |
| | | 12 M | 101.0 | 101.6 | 04:15 | 2% |
| | | 18 M | 99.9 | 100.8 | 03:55 | 2% |
| | | 24 M | 101.2 | 105.1 | 03:56 | 2% |
| S45000321A | 14 | 0 M | 98.0 | 101.7 | 05:22 | 3% |
| | | 3 M | 99.8 | 103.4 | 05:03 | 1% |
| | | 4 M | 98.8 | 101.4 | 05:08 | 1% |
| | | 5 M | 98.8 | 100.2 | 04:31 | 1% |
| | | 6 M | 96.8 | 99.9 | 04:16 | 1% |
| | | 9 M | 101.8 | 100.1 | 02:54 | 2% |
| | | 12 M | 101.6 | 101.8 | 03:20 | 1% |
| | | 18 M | 100.7 | 101.6 | 04:37 | 1% |
| | | 24 M | 102.8 | 108.1 | 03:37 | 2% |
| S45000321B | 90 | 0 M | 98.0 | 101.7 | 05:22 | 3% |
| | | 3 M | 98.5 | 101.7 | 05:28 | 1% |
| | | 4 M | 98.8 | 100.2 | 05:02 | 1% |
| | | 5 M | 98.3 | 100.6 | 05:03 | 1% |
| | | 6 M | 95.7 | 99.5 | 04:14 | 2% |
| | | 9 M | 99.9 | 99.3 | 04:08 | 2% |
| | | 12 M | 100.8 | 101.3 | 04:14 | 2% |
| | | 18 M | 98.9 | 105.5 | 04:12 | 2% |
| | | 24 M | 102.0 | 106.1 | 03:52 | 2% |

With reference to the results shown in Table 8, the present inventors surprisingly observed that oral solid dosage forms produced in Batches S45000121A, S45000121B, S45000221A, S45000221B, S45000321A, and S45000321B retained 100% of the original content of levothyroxine when stored under standard testing conditions at 25° C.±2° C. and 60%±5% relative humidity for a period of twenty-four months.

With reference to the results shown in Table 8, the present inventors also surprisingly observed that oral solid dosage forms produced in Batches S45000121A, S45000121B, S45000221A, S45000221B, S45000321A, and S45000321B retained 100% of the original content of liothyronine when stored under standard testing conditions at 25° C.±2° C. and 60%±5% relative humidity for a period of twenty-four months.

Intermediate condition stability samples were placed upright in an environmental chamber and maintained in an upright position during the stability testing. As discussed, each tablet was disposed in the sealed container containing either 10 to 20 or 90 to 100 tablets from Batches S45000121A, S45000121B, S45000221A, S45000221B, S45000321A, and S45000321B, and each tablet included about 9.5 μg of levothyroxine and about 2.2 μg of liothyronine per tablet. The sealed containers were maintained at 30° C.±2° C. and 65%±5% relative humidity, uninterrupted, (except for the addition or withdrawal of test samples) for a period of three months or six months. Tablets were removed from the storage conditions at the specified time points and tested for levothyroxine and liothyronine content using high pressure liquid chromatography.

Considering levothyroxine content, shelf life of tablets of Batches S45000121A, S45000121B, S45000221A, S45000221B, S45000321A, and S45000321B was estimated to be at least six months when stored under intermediate testing conditions. Table 9 provides the six-month stability assay results for tablets of Batches S45000121A, S45000121B, S45000221A, S45000221B, S45000321A, and S45000321B stored under intermediate testing conditions. The stability assay showed that greater than 95 percent of the original levothyroxine content was retained in tablets stored under intermediate testing conditions for six months.

Considering liothyronine content, shelf life of tablets of Batches S45000121A, S45000121B, S45000221A, S45000221B, S45000321A, and S45000321B was estimated to be at least six months when stored under intermediate testing conditions. As shown in Table 9, the stability assay showed that greater than 95 percent of the original liothyronine content was retained in tablets stored under intermediate testing conditions for six months.

TABLE 9
Stability assay results under intermediate testing conditions for tablets of Batches S45000121A,
S45000121B, S45000221A, S45000221B, S45000321A, and S45000321B
Thyroid Tablets, USP, 30° C./65% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 16.25 mg

| | | | | | | |
|---|---|---|---|---|---|---|
| S45000121A | 14 | 0 M | 97.0 | 100.4 | 05:01 | 3% |
| | | 3 M | 98.5 | 100.9 | 05:36 | 1% |
| | | 6 M | 97.6 | 103.1 | 05:17 | 1% |
| S45000121B | 90 | 0 M | 97.0 | 100.4 | 05:01 | 3% |
| | | 3 M | 99.2 | 101.1 | 05:35 | 1% |
| | | 6 M | 97.0 | 101.4 | 04:01 | 2% |
| S45000221A | 14 | 0 M | 98.6 | 103.2 | 04:34 | 3% |
| | | 3 M | 99.5 | 103.0 | 05:54 | 1% |
| | | 6 M | 97.2 | 101.9 | 04:23 | 1% |
| S45000221B | 90 | 0 M | 98.6 | 103.2 | 04:34 | 3% |
| | | 3 M | 100.6 | 104.3 | 05:24 | 1% |
| | | 6 M | 97.0 | 101.6 | 04:24 | 2% |
| S45000321A | 14 | 0 M | 98 | 101.7 | 05:22 | 3% |
| | | 3 M | 99.3 | 102.6 | 04:55 | 1% |
| | | 6 M | 97.3 | 102.0 | 04:36 | 1% |
| S45000321B | 90 | 0 M | 98.0 | 101.7 | 05:22 | 3% |
| | | 3 M | 100.4 | 103.7 | 05:13 | 1% |
| | | 6 M | 98.4 | 101.5 | 04:19 | 2% |

With reference to the results shown in Table 9, the present inventors surprisingly observed that oral solid dosage forms produced in Batches S45000121A, S45000121B, S45000221A, S45000221B, S45000321A, and S45000321B retained at least 98% of the original content of levothyroxine when stored under intermediate testing conditions at 30° C.±2° C. and 65%±5% relative humidity for a period of six months.

With reference to the results shown in Table 9, the present inventors also surprisingly observed that oral solid dosage forms produced in Batches S45000121A, S45000121B, S45000221A, S45000221B, S45000321A, and S45000321B retained at least 98% of the original content of liothyronine when stored under intermediate testing conditions at 30° C.±2° C. and 65%±5% relative humidity for a period of six months.

Accelerated condition stability samples were placed upright in an environmental chamber and maintained in an upright position during the stability testing. As discussed, each tablet was disposed in the sealed container containing either 10 to 20 or 90 to 100 tablets from Batches S45000121A, S45000121B, S45000221A, S45000221B, S45000321A, and S45000321B, and each tablet included about 9.5 μg of levothyroxine and about 2.2 μg of liothyronine per tablet. The sealed containers were maintained at 40° C.±2° C. and 75%±5% relative humidity, uninterrupted, (except for the addition or withdrawal of test samples) for a period of 1 month, 2 months, 3 months, 4 months, 5 months or six months. Tablets were removed from the storage conditions at the specified time points and tested for levothyroxine and liothyronine content using high pressure liquid chromatography.

Considering levothyroxine content, shelf life of tablets of Batches S45000121A, S45000121B, S45000221A, S45000221B, S45000321A, and S45000321B was estimated to be at least six months when stored under accelerated testing conditions. Table 10 provides the six-month stability assay results for tablets of Batches S45000121A, S45000121B, S45000221A, S45000221B, S45000321A, and S45000321B stored under accelerated testing conditions. The stability assay showed that greater than 95 percent of the original levothyroxine content was retained in tablets stored under accelerated testing conditions for six months.

Considering liothyronine content, shelf life of tablets of Batches S45000121A, S45000121B, S45000221A, S45000221B, S45000321A, and S45000321B was estimated to be at least six months when stored under accelerated testing conditions. As shown in Table 10, the stability assay showed that greater than 95 percent of the original liothyronine content was retained in tablets stored under accelerated testing conditions for six months.

TABLE 10
Stability assay results under accelerated testing conditions for tablets of Batches S45000121A,
S45000121B, S45000221A, S45000221B, S45000321A, and S45000321B
Thyroid Tablets, USP, 40 C./75% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 16.25 mg

| | | | | | | |
|---|---|---|---|---|---|---|
| S45000121A | 14 | 0 M | 97.0 | 100.4 | 05:01 | 3% |
| | | 1 M | 98.5 | 102.3 | 05:26 | 3% |
| | | 2 M | 98.3 | 103.4 | 05:20 | 1% |
| | | 3 M | 99.2 | 103.3 | 04:53 | 1% |
| | | 4 M | 99.7 | 102.5 | 04:57 | 2% |
| | | 5 M | 98.1 | 104.6 | 04:42 | 1% |
| | | 6 M | 95.4 | 102.2 | 04:19 | 2% |
| S45000121B | 90 | 0 M | 97.0 | 100.4 | 05:01 | 3% |
| | | 1 M | 98.8 | 102.5 | 05:36 | 3% |
| | | 2 M | 96.8 | 98.8 | 05:25 | 1% |
| | | 3 M | 99.1 | 102.7 | 04:52 | 1% |
| | | 4 M | 98.2 | 98.6 | 05:00 | 2% |
| | | 5 M | 97.6 | 98.4 | 05:28 | 2% |
| | | 6 M | 97.5 | 102.2 | 05:20 | 2% |
| S45000221A | 14 | 0 M | 98.6 | 103.2 | 04:34 | 3% |
| | | 1 M | 98.7 | 102.4 | 05:09 | 3% |
| | | 2 M | 100.0 | 104.8 | 05:11 | 1% |
| | | 3 M | 100.9 | 105.1 | 04:52 | 1% |
| | | 4 M | 98.2 | 100.6 | 05:07 | 2% |
| | | 5 M | 98.6 | 105.0 | 05:07 | 1% |
| | | 6 M | 97.8 | 104.6 | 04:42 | 2% |
| S45000221B | 90 | 0 M | 98.6 | 103.2 | 04:34 | 3% |
| | | 1 M | 98.9 | 103.0 | 05:25 | 3% |
| | | 2 M | 99.2 | 102.5 | 04:34 | 1% |
| | | 3 M | 99.2 | 102.3 | 06:19 | 1% |
| | | 4 M | 98.8 | 99.5 | 05:07 | 2% |
| | | 5 M | 97.4 | 97.1 | 04:52 | 2% |
| | | 6 M | 95.9 | 100.0 | 04:44 | 2% |
| S45000321A | 14 | 0 M | 98.0 | 101.7 | 05:22 | 3% |
| | | 1 M | 98.1 | 102.2 | 05:06 | 3% |
| | | 2 M | 100.7 | 105.6 | 04:53 | 1% |
| | | 3 M | 99.9 | 104.5 | 04:50 | 1% |
| | | 4 M | 98.3 | 100.6 | 05:11 | 2% |
| | | 5 M | 98.2 | 104.0 | 04:59 | 1% |
| | | 6 M | 96.1 | 102.9 | 04:09 | 2% |
| S45000321B | 90 | 0 M | 98.0 | 101.7 | 05:22 | 3% |
| | | 1 M | 98.3 | 102.3 | 05:06 | 3% |
| | | 2 M | 99.3 | 101.2 | 04:43 | 1% |
| | | 3 M | 100.0 | 101.9 | 05:41 | 1% |
| | | 4 M | 98.9 | 99.4 | 04:28 | 2% |
| | | 5 M | 98.0 | 99.6 | 05:12 | 2% |
| | | 6 M | 96.3 | 100.7 | 04:15 | 2% |

With reference to the results shown in Table 10, the present inventors surprisingly observed that oral solid dosage forms produced in Batches S45000121A, S45000121B, S45000221A, S45000221B, S45000321A, and S45000321B retained at least about 97% of the original content of levothyroxine when stored under accelerated testing conditions at 40° C.±2° C. and 75%±5% relative humidity for a period of six months.

With reference to the results shown in Table 10, the present inventors also surprisingly observed that oral solid dosage forms produced in Batches S45000121A, S45000121B, S45000221A, S45000221B, S45000321A, and S45000321B retained at least 97% of the original content of liothyronine when stored under accelerated testing conditions at 40° C.±2° C. and 75%±5% relative humidity for a period of six months.

### Example 6

A study was conducted to assess the stability of embodiments of oral solid dosage forms according to the present disclosure.

Tablets including 32.5 mg of porcine thyroid powder were prepared by compressing portions of several uniform mixtures made using the method generally described in Example 1. Each of the mixtures included porcine thyroid powder, microcrystalline cellulose (MCC), sodium starch glycolate, colloidal silicon dioxide, and calcium stearate. The MCC was included as a component of Avicel® HFE 102 powder. Mannitol was also a component of the Avicel® HFE 102 powder. Table 11 below provides the mass and weight/weight concentration of the ingredients in individual tablets comprising 32.5 mg porcine thyroid powder per tablet produced in Batch S45100121B.

TABLE 11
Composition of tablets in Batch S45100121B

| Ingredient | % w/w | mg/unit |
|---|---|---|
| Porcine thyroid powder, USP | 50.00 | 32.5 |
| Avicel ® HFE 102 powder | 44.15 | 28.7 |
| Sodium Starch Glycolate (SSG) | 2.54 | 1.65 |
| Colloidal Silicon Dioxide | 2.50 | 1.63 |
| Calcium Stearate | 0.81 | 0.53 |
| Total | 100.00 | 65.0 |

The 32.5 mg of porcine thyroid powder in each tablet comprised about 19 μg of levothyroxine and about 4.5 μg of liothyronine. Solid dosage forms including different amounts of the active agents may be formed in a dose similar manner by adjusting the amount of the active ingredient, along with the amount of inactive ingredients to keep the mass of the dosage forms the same.

For purposes of conducting the stability study, the tablets of Batch S45100121B were disposed in container closure systems including a container comprising an internal volume of 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed. As an alternative, air may be displaced within a container by disposing an inert pill packing material, for example, rayon or cotton, in the container after tablet loading and before induction sealing.

Three studies were conducted to assess the stability of the levothyroxine and liothyronine in the tablets produced in Batch S45100121B. The stability studies involved the following testing periods and conditions:
Storing under standard testing conditions (25° C.±2° C. and 60%±5% relative humidity) for up to 24 months.
Storing under intermediate testing conditions (30° C.±2° C. and 65%±5% relative humidity) for up to 6 months.
Storing under accelerated testing conditions (40° C.±2° C. and 75%±5% relative humidity) for up to 6 months.

A standard testing conditions stability study of the tablets of Batch S45100121B contained in the container closure system was conducted over a period of twenty-four months to determine the rate of physical or chemical degradation of the levothyroxine and liothyronine included in tablets when stored under the standard environmental conditions.

Container closure systems prepared as described above containing tablets including about 19 μg of levothyroxine and about 4.5 μg of liothyronine per tablet were placed upright in a calibrated environmental chamber and maintained in an upright position during the stability study. The sealed containers including 90 to 100 tablets were maintained at 25° C.±2° C. and 60%±5% relative humidity, uninterrupted (except for the addition or withdrawal of test samples), for a period of three months, four months, five months, six months, nine months, twelve months, eighteen months, or twenty-four months. Tablets were removed from the storage conditions at the specified time points and tested for levothyroxine and liothyronine content using high pressure liquid chromatography.

Considering levothyroxine content, shelf life of tablets of Batch S45100121B was estimated to be at least twenty-four months when stored under standard testing conditions. Table 12 provides the twenty-four month stability assay results for tablets of Batch S45100121B. The stability assay showed that greater than 95 percent of the original levothyroxine content was retained in tablets stored under standard testing conditions for twenty-four months.

Considering liothyronine content, shelf life of tablets of Batch S45100121B was estimated to be at least twenty-four months when stored under standard testing conditions. As shown in Table 12, the stability assay showed that greater than 95 percent of the original liothyronine content was retained in tablets stored under standard testing conditions for twenty-four months.

TABLE 12
Stability assay results under standard testing conditions for tablets of Batch S4500121B
Thyroid Tablets, USP 25° C./60% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 32.5 mg

| | | | | | | |
|---|---|---|---|---|---|---|
| S45100121B | 90 | 0 M | 97.9 | 102 | 04:37 | 3% |
| | | 3 M | 100.8 | 104.3 | 05:00 | 1% |
| | | 4 M | 99.8 | 100.7 | 04:20 | 1% |
| | | 5 M | 98.9 | 100.7 | 04:54 | 1% |
| | | 6 M | 95.8 | 98.9 | 04:08 | 2% |
| | | 9 M | 101.6 | 100.1 | 04:08 | 2% |
| | | 12 M | 101.4 | 103.5 | 04:04 | 2% |
| | | 18 M | 98.4 | 104.8 | 04:45 | 2% |
| | | 24 M | 101.3 | 105.6 | 04:14 | 2% |

With reference to the results shown in Table 12, the present inventors surprisingly observed that oral solid dosage forms produced in Batch S45100121B retained about 100% of the original content of levothyroxine when stored under standard testing conditions at 25° C.±2° C. and 60%±5% relative humidity for a period of twenty-four months.

With reference to the results shown in Table 12, the present inventors also surprisingly observed that oral solid dosage forms produced in Batch S45100121B retained about 100% of the original content of liothyronine when stored under standard testing conditions at 25° C.±2° C. and 60%±5% relative humidity for a period of twenty-four months.

Intermediate condition stability samples were placed upright in an environmental chamber and maintained in an upright position during the stability testing. As discussed, each tablet was disposed in the sealed container containing 90 to 100 tablets from Batch S45100121B and each tablet included about 19 μg of levothyroxine and about 4.5 μg of liothyronine per tablet. The sealed containers were maintained at 30° C.±2° C. and 65%±5% relative humidity, uninterrupted, (except for the addition or withdrawal of test samples) for a period of three months or six months. Tablets were removed from the storage conditions at the specified time points and tested for levothyroxine and liothyronine content using high pressure liquid chromatography.

Considering levothyroxine content, shelf life of tablets of Batch S45100121B was estimated to be at least six months when stored under intermediate testing conditions. Table 13 provides the six-month stability assay results for tablets of Batch S45100121B stored under intermediate testing conditions. The stability assay showed that greater than 95 percent of the original levothyroxine content was retained in tablets stored under intermediate testing conditions for six months.

Considering liothyronine content, shelf life of tablets of Batch S45100121B was estimated to be at least six months when stored under intermediate testing conditions. As shown in Table 13, the stability assay showed that greater than 95 percent of the original liothyronine content was retained in tablets stored under intermediate testing conditions for six months.

TABLE 13
Stability assay results under intermediate testing conditions for tablets of Batch S45100121B
Thyroid Tablets, USP, 30° C./65% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 32.5 mg

| | | | | | | |
|---|---|---|---|---|---|---|
| S45100121B | 90 | 0 M | 97.9 | 102.0 | 04:37 | 3% |
| | | 3 M | 100.4 | 104.0 | 05:25 | 1% |
| | | 6 M | 97.2 | 100.5 | 04:49 | 2% |

With reference to the results shown in Table 13, the present inventors surprisingly observed that oral solid dosage forms produced in Batch S45100121B retained 98.5% of the original content of levothyroxine when stored under intermediate testing conditions at 30° C.±2° C. and 65%±5% relative humidity for a period of six months.

With reference to the results shown in Table 13, the present inventors also surprisingly observed that oral solid dosage forms produced in Batch S45100121B retained 99% of the original content of liothyronine when stored under intermediate testing conditions at 30° C.±2° C. and 65%±5% relative humidity for a period of six months.

Accelerated condition stability samples were placed upright in an environmental chamber and maintained in an upright position during the stability testing.

As discussed, each tablet was disposed in the sealed container containing 90 to 100 tablets from Batch S45100121B and each tablet included about 19 μg of levothyroxine and about 4.5 μg of liothyronine per tablet. The sealed containers were maintained at 40° C.±2° C. and 75%±5% relative humidity, uninterrupted, (except for the addition or withdrawal of test samples) for a period of 1 month, 2 months, 3 months, 4 months, 5 months or six months. Tablets were removed from the storage conditions at the specified time points and tested for levothyroxine and liothyronine content using high pressure liquid chromatography.

Considering levothyroxine content, shelf life of tablets of Batch S45100121B was estimated to be at least six months when stored under accelerated testing conditions. Table 14 provides the six-month stability assay results for tablets of Batch S45100121B stored under accelerated testing conditions. The stability assay showed that greater than 95 percent of the original levothyroxine content was retained in tablets stored under accelerated testing conditions for six months.

Considering liothyronine content, shelf life of tablets of Batch S45100121B was estimated to be at least six months when stored under accelerated testing conditions. As shown in Table 14, the stability assay showed that greater than 95 percent of the original liothyronine content was retained in tablets stored under accelerated testing conditions for six months.

TABLE 14
Stability assay results under accelerated testing conditions for tablets of Batch S45100121B
Thyroid Tablets, USP, 40 C./75% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 32.5 mg

| | | | | | | |
|---|---|---|---|---|---|---|
| S45100121B | 90 | 0 M | 97.9 | 102.0 | 04:37 | 3% |
| | | 1 M | 99.5 | 103.2 | 05:30 | 3% |
| | | 2 M | 99.0 | 101.7 | 05:05 | 1% |
| | | 3 M | 100 | 101.3 | 04:59 | 2% |
| | | 4 M | 100.3 | 100.6 | 05:16 | 2% |
| | | 5 M | 97.5 | 99.2 | 05:48 | 2% |
| | | 6 M | 96.1 | 100.3 | 04:32 | 2% |

With reference to the results shown in Table 14, the present inventors surprisingly observed that oral solid dosage forms produced in Batch S45100121B retained about 98% of the original content of levothyroxine when stored under accelerated testing conditions at 40° C.±2° C. and 75%±5% relative humidity for a period of six months.

With reference to the results shown in Table 14, the present inventors also surprisingly observed that oral solid dosage forms produced in Batch S45100121B retained about 98% of the original content of liothyronine when stored under accelerated testing conditions at 40° C.±2° C. and 75%±5% relative humidity for a period of six months.

### Example 7

A study was conducted to assess the stability of embodiments of oral solid dosage forms according to the present disclosure.

Tablets including 65 mg of porcine thyroid powder were prepared by compressing portions of several uniform mixtures made using the method generally described in Example 1. Each of the mixtures included porcine thyroid powder, microcrystalline cellulose (MCC), sodium starch glycolate, colloidal silicon dioxide, and calcium stearate. The MCC was included as a component of Avicel® HFE 102 powder. Mannitol was also a component of the Avicel® HFE 102 powder. Table 15 below provides the mass and weight/weight concentration of the ingredients in individual tablets comprising 65 mg porcine thyroid powder per tablet produced in Batch S45200121A.

TABLE 15
Composition of tablets in Batch S45200121A

| Ingredient | % w/w | mg/unit |
|---|---|---|
| Porcine thyroid powder, USP | 50.00 | 65.0 |
| Avicel ® HFE 102 powder | 44.15 | 57.4 |
| Sodium Starch Glycolate (SSG) | 2.54 | 3.3 |
| Colloidal Silicon Dioxide | 2.50 | 3.25 |
| Calcium Stearate | 0.81 | 1.05 |
| Total | 100.00 | 130.0 |

The 65 mg of porcine thyroid powder in each tablet comprised about 38 μg of levothyroxine and about 9 μg of liothyronine. Solid dosage forms including different amounts of the active agents may be formed in a dose similar manner by adjusting the amount of the active ingredient, along with the amount of inactive ingredients to keep the mass of the dosage forms the same.

For purposes of conducting the stability study, the tablets of Batch S45200121A were disposed in container closure systems including a container comprising an internal volume of 30 cm³ (including 10 to 20 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed. As an alternative, air may be displaced within a container by disposing an inert pill packing material, for example, rayon or cotton, in the container after tablet loading and before induction sealing.

Three studies were conducted to assess the stability of the levothyroxine and liothyronine in the tablets produced in Batch S45200121A. The stability studies involved the following testing periods and conditions:
Storing under standard testing conditions (25° C.±2° C. and 60%±5% relative humidity) for up to 24 months.
Storing under intermediate testing conditions (30° C.±2° C. and 65%±5% relative humidity) for up to 6 months.
Storing under accelerated testing conditions (40° C.±2° C. and 75%±5% relative humidity) for up to 6 months.

A standard testing conditions stability study of the tablets of Batch S45200121A contained in the container closure system was conducted over a period of twenty-four months to determine the rate of physical or chemical degradation of the levothyroxine and liothyronine included in tablets when stored under the standard environmental conditions.

Container closure systems prepared as described above containing tablets including about 38 μg of levothyroxine and about 9 μg of liothyronine per tablet were placed upright in a calibrated environmental chamber and maintained in an upright position during the stability study. The sealed containers including 10 to 20 tablets were maintained at 25° C.±2° C. and 60%±5% relative humidity, uninterrupted (except for the addition or withdrawal of test samples), for a period of three months, four months, five months, six months, nine months, twelve months, eighteen months, or twenty-four months. Tablets were removed from the storage conditions at the specified time points and tested for levothyroxine and liothyronine content using high pressure liquid chromatography.

Considering levothyroxine content, shelf life of tablets of Batch S45200121A was estimated to be at least twenty-four months when stored under standard testing conditions. Table 16 provides the twenty-four month stability assay results for tablets of Batch S45200121A. The stability assay showed that greater than 95 percent of the original levothyroxine content was retained in tablets stored under standard testing conditions for twenty-four months.

Considering liothyronine content, shelf life of tablets of Batch S45200121A was estimated to be at least twenty-four months when stored under standard testing conditions. As shown in Table 16, the stability assay showed that greater than 95 percent of the original liothyronine content was retained in tablets stored under standard testing conditions for twenty-four months.

TABLE 16
Stability assay results under standard testing conditions for tablets of Batch S45200121A
Thyroid Tablets, USP 25° C./60% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 65 mg

| | | | | | | |
|---|---|---|---|---|---|---|
| S45200121A | 14 | 0 M | 97.7 | 101.8 | 05:04 | 3% |
| | | 3 M | 100.0 | 103.5 | 05:22 | 1% |
| | | 4 M | 101.4 | 101.4 | 05:53 | 1% |
| | | 5 M | 99.6 | 101.8 | 05:18 | 1% |
| | | 6 M | 95.6 | 100.3 | 05:16 | 2% |
| | | 9 M | 100.1 | 98.9 | 03:31 | 3% |
| | | 12 M | 97.9 | 103.8 | 04:29 | 2% |
| | | 18 M | 99.3 | 102.5 | 05:04 | 1% |
| | | 24 M | 101.5 | 105.8 | 04:50 | 2% |

With reference to the results shown in Table 16, the present inventors surprisingly observed that oral solid dosage forms produced in Batch S45200121A retained about 100% of the original content of levothyroxine when stored under standard testing conditions at 25° C.±2° C. and 60%±5% relative humidity for a period of twenty-four months.

With reference to the results shown in Table 16, the present inventors also surprisingly observed that oral solid dosage forms produced in Batch S45200121A retained about 100% of the original content of liothyronine when stored under standard testing conditions at 25° C.±2° C. and 60%±5% relative humidity for a period of twenty-four months.

Intermediate condition stability samples were placed upright in an environmental chamber and maintained in an upright position during the stability testing. As discussed, each tablet was disposed in the sealed container containing 10 to 20 tablets from Batch S45200121A and each tablet included about 38 μg of levothyroxine and about 9 μg of liothyronine per tablet. The sealed containers were maintained at 30° C.±2° C. and 65%±5% relative humidity, uninterrupted, (except for the addition or withdrawal of test samples) for a period of three months or six months. Tablets were removed from the storage conditions at the specified time points and tested for levothyroxine and liothyronine content using high pressure liquid chromatography.

Considering levothyroxine content, shelf life of tablets of Batch S45200121A was estimated to be at least six months when stored under intermediate testing conditions. Table 17 provides the six-month stability assay results for tablets of Batch S45200121A stored under intermediate testing conditions. The stability assay showed that greater than 95 percent of the original levothyroxine content was retained in tablets stored under intermediate testing conditions for six months.

Considering liothyronine content, shelf life of tablets of Batch S45200121A was estimated to be at least six months when stored under intermediate testing conditions. As shown in Table 17, the stability assay showed that greater than 95 percent of the original liothyronine content was retained in tablets stored under intermediate testing conditions for six months.

TABLE 17
Stability assay results under intermediate testing conditions for tablets of Batch S45200121A
Thyroid Tablets, USP, 30° C./65% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 65 mg

| | | | | | | |
|---|---|---|---|---|---|---|
| S45200121A | 14 | 0 M | 97.7 | 101.8 | 05:04 | 3% |
| | | 3 M | 100.6 | 104.0 | 05:09 | 1% |
| | | 6 M | 98.8 | 103.6 | 05:22 | 2% |

With reference to the results shown in Table 17, the present inventors surprisingly observed that oral solid dosage forms produced in Batch S45200121A retained 100% of the original content of levothyroxine when stored under intermediate testing conditions at 30° C.±2° C. and 65%±5% relative humidity for a period of six months.

With reference to the results shown in Table 17, the present inventors also surprisingly observed that oral solid dosage forms produced in Batch S45200121A retained 100% of the original content of liothyronine when stored under intermediate testing conditions at 30° C.±2° C. and 65%±5% relative humidity for a period of six months.

Accelerated condition stability samples were placed upright in an environmental chamber and maintained in an upright position during the stability testing. As discussed, each tablet was disposed in the sealed container containing 10 to 20 tablets from Batch S45200121A and each tablet included about 38 μg of levothyroxine and about 9 μg of liothyronine per tablet. The sealed containers were maintained at 40° C.±2° C. and 75%±5% relative humidity, uninterrupted, (except for the addition or withdrawal of test samples) for a period of 1 month, 2 months, 3 months, 4 months, 5 months or six months. Tablets were removed from the storage conditions at the specified time points and tested for levothyroxine and liothyronine content using high pressure liquid chromatography.

Considering levothyroxine content, shelf life of tablets of Batch S45200121A was estimated to be at least six months when stored under accelerated testing conditions. Table 18 provides the six-month stability assay results for tablets of Batch S45200121A stored under accelerated testing conditions. The stability assay showed that greater than 95 percent of the original levothyroxine content was retained in tablets stored under accelerated testing conditions for six months.

Considering liothyronine content, shelf life of tablets of Batch S45200121A was estimated to be at least six months when stored under accelerated testing conditions. As shown in Table 18, the stability assay showed that greater than 95 percent of the original liothyronine content was retained in tablets stored under accelerated testing conditions for six months.

TABLE 18
Stability assay results under accelerated testing conditions for tablets of Batch S45200121A
Thyroid Tablets, USP, 40 C./75% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 65 mg

| | | | | | | |
|---|---|---|---|---|---|---|
| S45200121A | 14 | 0 M | 97.7 | 101.8 | 05:04 | 3% |
| | | 1 M | 97.8 | 101.5 | 05:57 | 2% |
| | | 2 M | 97.4 | 101.1 | 05:18 | 1% |
| | | 3 M | 99.5 | 102.8 | 05:33 | 1% |
| | | 4 M | 100.7 | 99.6 | 05:39 | 1% |
| | | 5 M | 97.4 | 99.3 | 04:44 | 2% |
| | | 6 M | 95.6 | 102.0 | 05:33 | 2% |

With reference to the results shown in Table 18, the present inventors surprisingly observed that oral solid dosage forms produced in Batch S45200121A retained 100% of the original content of levothyroxine when stored under accelerated testing conditions at 40° C.±2° C. and 75%±5% relative humidity for a period of six months.

With reference to the results shown in Table 18, the present inventors also surprisingly observed that oral solid dosage forms produced in Batch S45200121A retained about 98% of the original content of liothyronine when stored under accelerated testing conditions at 40° C.±2° C. and 75%±5% relative humidity for a period of six months.

### Example 8

A study was conducted to assess the stability of embodiments of oral solid dosage forms according to the present disclosure.

Tablets including 97.5 mg of porcine thyroid powder were prepared by compressing portions of several uniform mixtures made using the method generally described in Example 1. Each of the mixtures included porcine thyroid powder, microcrystalline cellulose (MCC), sodium starch glycolate, colloidal silicon dioxide, and calcium stearate. The MCC was included as a component of Avicel® HFE 102 powder. Mannitol was also a component of the Avicel® HFE 102 powder. Table 19 below provides the mass and weight/weight concentration of the ingredients in individual tablets comprising 97.5 mg porcine thyroid powder per tablet produced in Batch S45300121B.

TABLE 19
Composition of tablets in Batch S45300121B

| Ingredient | % w/w | mg/unit |
|---|---|---|
| Porcine thyroid powder, USP | 50.00 | 97.5 |
| Avicel ® HFE 102 powder | 44.15 | 86.09 |
| Sodium Starch Glycolate (SSG) | 2.54 | 4.95 |
| Colloidal Silicon Dioxide | 2.50 | 4.88 |
| Calcium Stearate | 0.81 | 1.58 |
| Total | 100.00 | 195.0 |

The 97.5 mg of porcine thyroid powder in each tablet comprised about 57 μg of levothyroxine and about 13.5 μg of liothyronine. Solid dosage forms including different amounts of the active agents may be formed in a dose similar manner by adjusting the amount of the active ingredient, along with the amount of inactive ingredients to keep the mass of the dosage forms the same.

For purposes of conducting the stability study, the tablets of Batch S45300121B were disposed in container closure systems including a container comprising an internal volume of 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed. As an alternative, air may be displaced within a container by disposing an inert pill packing material, for example, rayon or cotton, in the container after tablet loading and before induction sealing.

Three studies were conducted to assess the stability of the levothyroxine and liothyronine in the tablets produced in Batch S453001218. The stability studies involved the following testing periods and conditions:
Storing under standard testing conditions (25° C.±2° C. and 60%±5% relative humidity) for up to 24 months.
Storing under intermediate testing conditions (30° C.±2° C. and 65%±5% relative humidity) for up to 6 months.
Storing under accelerated testing conditions (40° C.±2° C. and 75%±5% relative humidity) for up to 6 months.

A standard testing conditions stability study of the tablets of Batch S45300121B contained in the container closure system was conducted over a period of twenty-four months to determine the rate of physical or chemical degradation of the levothyroxine and liothyronine included in tablets when stored under the standard environmental conditions.

Container closure systems prepared as described above containing tablets including about 57 μg of levothyroxine and about 13.5 μg of liothyronine per tablet were placed upright in a calibrated environmental chamber and maintained in an upright position during the stability study. The sealed containers including 90 to 100 tablets were maintained at 25° C.±2° C. and 60%±5% relative humidity, uninterrupted (except for the addition or withdrawal of test samples), for a period of three months, four months, five months, six months, nine months, twelve months, eighteen months, or twenty-four months. Tablets were removed from the storage conditions at the specified time points and tested for levothyroxine and liothyronine content using high pressure liquid chromatography.

Considering levothyroxine content, shelf life of tablets of Batch S45300121B was estimated to be at least twenty-four months when stored under standard testing conditions. Table 20 provides the twenty-four month stability assay results for tablets of Batch S45300121B. The stability assay showed that greater than 95 percent of the original levothyroxine content was retained in tablets stored under standard testing conditions for twenty-four months.

Considering liothyronine content, shelf life of tablets of Batch S45300121B was estimated to be at least twenty-four months when stored under standard testing conditions. As shown in Table 20, the stability assay showed that greater than 95 percent of the original liothyronine content was retained in tablets stored under standard testing conditions for twenty-four months.

TABLE 20
Stability assay results under standard testing conditions for tablets of Batch S45300121B
Thyroid Tablets, USP 25° C./60% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 97.5 mg

| | | | | | | |
|---|---|---|---|---|---|---|
| S45300121B | 90 | 0 M | 97.9 | 101.9 | 04:45 | 3% |
| | | 3 M | 100.2 | 104.0 | 05:05 | 2% |
| | | 4 M | 102.1 | 102.5 | 05:47 | 2% |
| | | 5 M | 98.4 | 101.0 | 04:48 | 2% |
| | | 6 M | 94.6 | 97.7 | 05:25 | 2% |
| | | 9 M | 97.2 | 101.2 | 04:11 | 2% |
| | | 12 M | 97.3 | 101.4 | 05:08 | 2% |
| | | 18 M | 99.5 | 105.4 | 04:33 | 2% |
| | | 24 M | 101.2 | 105.0 | 05:11 | 2% |

With reference to the results shown in Table 20, the present inventors surprisingly observed that oral solid dosage forms produced in Batch S45300121B retained 100% of the original content of levothyroxine when stored under standard testing conditions at 25° C.±2° C. and 60%±5% relative humidity for a period of twenty-four months.

With reference to the results shown in Table 20, the present inventors also surprisingly observed that oral solid dosage forms produced in Batch S45300121B retained 100% of the original content of liothyronine when stored under standard testing conditions at 25° C.±2° C. and 60%±5% relative humidity for a period of twenty-four months.

Intermediate condition stability samples were placed upright in an environmental chamber and maintained in an upright position during the stability testing. As discussed, each tablet was disposed in the sealed container containing 90 to 100 tablets from Batch S45300121B and each tablet included about 57 μg of levothyroxine and about 13.5 μg of liothyronine per tablet. The sealed containers were maintained at 30° C.±2° C. and 65%±5% relative humidity, uninterrupted, (except for the addition or withdrawal of test samples) for a period of three months or six months. Tablets were removed from the storage conditions at the specified time points and tested for levothyroxine and liothyronine content using high pressure liquid chromatography.

Considering levothyroxine content, shelf life of tablets of Batch S45300121B was estimated to be at least six months when stored under intermediate testing conditions. Table 21 provides the six-month stability assay results for tablets of Batch S45300121B stored under intermediate testing conditions. The stability assay showed that greater than 95 percent of the original levothyroxine content was retained in tablets stored under intermediate testing conditions for six months.

Considering liothyronine content, shelf life of tablets of Batch S45300121B was estimated to be at least six months when stored under intermediate testing conditions. As shown in Table 21, the stability assay showed that greater than 95 percent of the original liothyronine content was retained in tablets stored under intermediate testing conditions for six months.

TABLE 21
Stability assay results under intermediate testing conditions for tablets of Batch S45300121B
Thyroid Tablets, USP, 30° C./65% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 97.5 mg

| | | | | | | |
|---|---|---|---|---|---|---|
| S45300121B | 90 | 0 M | 97.9 | 101.9 | 04:45 | 3% |
| | | 3 M | 98.7 | 100.8 | 05:39 | 2% |
| | | 6 M | 95.0 | 98.6 | 05:11 | 2% |

With reference to the results shown in Table 21, the present inventors surprisingly observed that oral solid dosage forms produced in Batch S45300121B retained about 97% of the original content of levothyroxine when stored under intermediate testing conditions at 30° C.±2° C. and 65%±5% relative humidity for a period of six months.

With reference to the results shown in Table 21, the present inventors also surprisingly observed that oral solid dosage forms produced in Batch S45300121B retained 97% of the original content of liothyronine when stored under intermediate testing conditions at 30° C.±2° C. and 65%±5% relative humidity for a period of six months.

Accelerated condition stability samples were placed upright in an environmental chamber and maintained in an upright position during the stability testing. As discussed, each tablet was disposed in the sealed container containing 90 to 100 tablets from Batch S45300121B and each tablet included about 57 μg of levothyroxine and about 13.5 μg of liothyronine per tablet. The sealed containers were maintained at 40° C.±2° C. and 75%±5% relative humidity, uninterrupted, (except for the addition or withdrawal of test samples) for a period of 1 month, 2 months, 3 months, 4 months, 5 months or six months. Tablets were removed from the storage conditions at the specified time points and tested for levothyroxine and liothyronine content using high pressure liquid chromatography.

Considering levothyroxine content, shelf life of tablets of Batch S45300121B was estimated to be at least six months when stored under accelerated testing conditions. Table 22 provides the six-month stability assay results for tablets of Batch S45300121B stored under accelerated testing conditions. The stability assay showed that greater than 95 percent of the original levothyroxine content was retained in tablets stored under accelerated testing conditions for six months.

Considering liothyronine content, shelf life of tablets of Batch S45300121B was estimated to be at least six months when stored under accelerated testing conditions. As shown in Table 22, the stability assay showed that greater than 95 percent of the original liothyronine content was retained in tablets stored under accelerated testing conditions for six months.

TABLE 22
Stability assay results under accelerated testing conditions for tablets of Batch S45300121B
Thyroid Tablets, USP, 40 C./75% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 97.5 mg

| | | | | | | |
|---|---|---|---|---|---|---|
| S45300121B | 90 | 0 M | 97.9 | 101.9 | 04:45 | 3% |
| | | 1 M | 99.7 | 103.3 | 05:18 | 2% |
| | | 2 M | 99.4 | 103.0 | 06:17 | 2% |
| | | 3 M | 99.9 | 102.9 | 05:50 | 2% |
| | | 4 M | 96.0 | 97.0 | 06:05 | 2% |
| | | 5 M | 98.0 | 101.2 | 06:35 | 2% |
| | | 6 M | 96.4 | 100.4 | 06:54 | 2% |

With reference to the results shown in Table 22, the present inventors surprisingly observed that oral solid dosage forms produced in Batch S45300121B retained 98.5% of the original content of levothyroxine when stored under accelerated testing conditions at 40° C.±2° C. and 75%±5% relative humidity for a period of six months.

With reference to the results shown in Table 22, the present inventors also surprisingly observed that oral solid dosage forms produced in Batch S45300121B retained 98% of the original content of liothyronine when stored under accelerated testing conditions at 40° C.±2° C. and 75%±5% relative humidity for a period of six months.

### Example 9

A study was conducted to assess the stability of embodiments of oral solid dosage forms according to the present disclosure.

Tablets including 130 mg of porcine thyroid powder were prepared by compressing portions of several uniform mixtures made using the method generally described in Example 1. Each of the mixtures included porcine thyroid powder, microcrystalline cellulose (MCC), sodium starch glycolate, colloidal silicon dioxide, and calcium stearate. The MCC was included as a component of Avicel® HFE 102 powder. Mannitol was also a component of the Avicel® HFE 102 powder. Table 23 below provides the mass and weight/weight concentration of the ingredients in individual tablets comprising 130 mg porcine thyroid powder per tablet produced in Batches S45400121A, S45400121B, S45400221A, S45400221B, S45400321A, and S45400321B.

TABLE 23
Composition of tablets in Batches S45400121A, S45400121B,
S45400221A, S45400221B, S45400321A, and S45400321B

| Ingredient | % w/w | mg/unit |
|---|---|---|
| Porcine thyroid powder, USP | 50.00 | 130.0 |
| Avicel ® HFE 102 powder | 44.15 | 114.79 |
| Sodium Starch Glycolate (SSG) | 2.54 | 6.6 |
| Colloidal Silicon Dioxide | 2.50 | 6.5 |
| Calcium Stearate | 0.81 | 2.11 |
| Total | 100.00 | 260.0 |

The 130 mg of porcine thyroid powder in each tablet comprised about 76 μg of levothyroxine and about 18 μg of liothyronine. Solid dosage forms including different amounts of the active agents may be formed in a dose similar manner by adjusting the amount of the active ingredient, along with the amount of inactive ingredients to keep the mass of the dosage forms the same.

For purposes of conducting the stability study, the tablets of Batches S45400121A, S45400121B, S45400221A, S45400221B, S45400321A, and S45400321B were disposed in container closure systems including a container comprising an internal volume of either about 30 cm³ (including 10 to 20 tablets) or about 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed. As an alternative, air may be displaced within a container by disposing an inert pill packing material, for example, rayon or cotton, in the container after tablet loading and before induction sealing.

Three studies were conducted to assess the stability of the levothyroxine and liothyronine in the tablets produced in Batches S45400121A, S45400121B, S45400221A, S45400221B, S45400321A, and S45400321B. The stability studies involved the following testing periods and conditions:
Storing under standard testing conditions (25° C. #2° C. and 60%±5% relative humidity) for up to 24 months.
Storing under intermediate testing conditions (30° C.±2° C. and 65%±5% relative humidity) for up to 6 months.
Storing under accelerated testing conditions (40° C.±2° C. and 75%±5% relative humidity) for up to 6 months.

A standard testing conditions stability study of the tablets of Batches S45400121A, S45400121B, S45400221A, S45400221B, S45400321A, and S45400321B contained in the container closure system was conducted over a period of twenty-four months to determine the rate of physical or chemical degradation of the levothyroxine and liothyronine included in tablets when stored under the standard environmental conditions.

Container closure systems prepared as described above containing tablets including about 76 μg of levothyroxine and about 18 μg of liothyronine per tablet were placed upright in a calibrated environmental chamber and maintained in an upright position during the stability study. The sealed containers including either 10 to 20 or 90 to 100 tablets were maintained at 25° C. #2° C. and 60%±5% relative humidity, uninterrupted (except for the addition or withdrawal of test samples), for a period of three months, four months, five months, six months, nine months, twelve months, eighteen months, or twenty-four months. Tablets were removed from the storage conditions at the specified time points and tested for levothyroxine and liothyronine content using high pressure liquid chromatography.

Considering levothyroxine content, shelf life of tablets of Batches S45400121A, S45400121B, S45400221A, S45400221B, S45400321A, and S45400321B was estimated to be at least twenty-four months when stored under standard testing conditions. Table 24 provides the twenty-four month stability assay results for tablets of Batches S45400121A, S45400121B, S45400221A, S45400221B, S45400321A, and S45400321B. The stability assay showed that greater than 95 percent of the original levothyroxine content was retained in tablets stored under standard testing conditions for twenty-four months.

Considering liothyronine content, shelf life of tablets of Batches S45400121A, S45400121B, S45400221A, S45400221B, S45400321A, and S45400321B was estimated to be at least twenty-four months when stored under standard testing conditions. As shown in Table 24, the stability assay showed that greater than 95 percent of the original liothyronine content was retained in tablets stored under standard testing conditions for twenty-four months.

TABLE 24
Stability assay results under standard testing conditions for tablets of Batches S45400121A,
S45400121B, S45400221A, S45400221B, S45400321A, and S45400321B
Thyroid Tablets, USP 25° C./60% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 130 mg

| | | | | | | |
|---|---|---|---|---|---|---|
| S45400121A | 14 | 0 M | 97.5 | 101.4 | 02:52 | 2% |
| | | 3 M | 99.6 | 102.8 | 05:04 | T% |
| | | 4 M | 97.6 | 98.4 | 04:10 | 1% |
| | | 5 M | 98.2 | 101.4 | 04:37 | 1% |
| | | 6 M | 94.8 | 98.2 | 04:33 | 1% |
| | | 9 M | 96.6 | 100.4 | 03:01 | 2% |
| | | 12 M | 96.6 | 100.5 | 02:49 | 2% |
| | | 18 M | 99.7 | 105.3 | 03:23 | 1% |
| | | 24 M | 101.0 | 105.4 | 04:21 | 2% |
| S45400121B | 90 | 0 M | 97.5 | 101.4 | 02:52 | 2% |
| | | 3 M | 99.0 | 102.3 | 04:55 | 2% |
| | | 4 M | 98.6 | 99.7 | 06:05 | 2% |
| | | 5 M | 98.9 | 102.3 | 03:59 | 2% |
| | | 6 M | 96.0 | 100.0 | 04:29 | 2% |
| | | 9 M | 99.3 | 04.3 | 03:27 | 2% |
| | | 12 M | 96.5 | 99.6 | 03:42 | 2% |
| | | 18 M | 97.7 | 103.7 | 04:23 | 2% |
| | | 24 M | 99.8 | 103.8 | 04:08 | 2% |
| S45400221A | 14 | 0 M | 97.9 | 101.1 | 04:10 | 2% |
| | | 3 M | 98.0 | 100.6 | 04:15 | 1% |
| | | 4 M | 97.5 | 100.8 | 07:10 | 1% |
| | | 5 M | 97.1 | 101.0 | 04:47 | 1% |
| | | 6 M | 95.2 | 99.3 | 03:56 | 1% |
| | | 9 M | 95.2 | 97.9 | 03:31 | 2% |
| | | 12 M | 95.9 | 99.5 | 03:38 | 2% |
| | | 18 M | 99.3 | 104.7 | 03:49 | 2% |
| | | 24 M | 99.7 | 103.8 | 03:54 | 2% |
| S45400221B | 90 | 0 M | 97.9 | 101.1 | 04:10 | 2% |
| | | 3 M | 99.2 | 102.2 | 04:14 | 2% |
| | | 4 M | 97.6 | 99.0 | 05:02 | 2% |
| | | 5 M | 98.1 | 99.8 | 04:33 | 2% |
| | | 6 M | 97.5 | 97.8 | 03:36 | 2% |
| | | 9 M | 99.2 | 104.0 | 04:03 | 3% |
| | | 12 M | 99.8 | 100.9 | 03:36 | 2% |
| | | 18 M | 99.6 | 105.7 | 04:50 | 2% |
| | | 24 M | 101.6 | 105.3 | 03:48 | 2% |
| S45400321A | 14 | 0 M | 97.5 | 101.5 | 03:21 | 3% |
| | | 3 M | 99.5 | 103.0 | 03:48 | 1% |
| | | 4 M | 98.3 | 100.8 | 04:55 | 1% |
| | | 5 M | 98.2 | 102.1 | 03:32 | 1% |
| | | 6 M | 95.1 | 99.5 | 03:16 | 2% |
| | | 9 M | 97.2 | 101.0 | 02:58 | 2% |
| | | 12 M | 97.4 | 100.3 | 02:28 | 2% |
| | | 18 M | 100.3 | 105.9 | 03:32 | 2% |
| | | 24 M | 100.2 | 104.4 | 02:44 | 2% |
| S45400321B | 90 | 0 M | 97.5 | 101.5 | 03:21 | 3% |
| | | 3 M | 98.7 | 101.6 | 03:34 | 2% |
| | | 4 M | 102.1 | 103.0 | 03:35 | 2% |
| | | 5 M | 98.0 | 99.6 | 03:39 | 2% |
| | | 6 M | 97.5 | 97.7 | 03:29 | 2% |
| | | 9 M | 99.3 | 103.9 | 04:01 | 3% |
| | | 12 M | 97.4 | 103.2 | 03:34 | 2% |
| | | 18 M | 99.1 | 105.2 | 03:45 | 2% |
| | | 24 M | 102.1 | 105.9 | 02:12 | 2% |

With reference to the results shown in Table 24, the present inventors surprisingly observed that oral solid dosage forms produced in Batches $45400121A, S45400121B, S45400221A, S45400221B, S45400321A, and S45400321B retained 100% of the original content of levothyroxine when stored under standard testing conditions at 25° C.±2° C. and 60%±5% relative humidity for a period of twenty-four months.

With reference to the results shown in Table 24, the present inventors also surprisingly observed that oral solid dosage forms produced in Batches S45400121A, S45400121B, S45400221A, S45400221B, S45400321A, and S45400321B retained 100% of the original content of liothyronine when stored under standard testing conditions at 25° C.±2° C. and 60%±5% relative humidity for a period of twenty-four months.

Intermediate condition stability samples were placed upright in an environmental chamber and maintained in an upright position during the stability testing. As discussed, each tablet was disposed in the sealed container containing either 10 to 20 or 90 to 100 tablets from Batches S45400121A, S45400121B, S45400221A, S45400221B, S45400321A, and S45400321B, and each tablet included about 76 μg of levothyroxine and about 18 μg of liothyronine per tablet. The sealed containers were maintained at 30° C.±2° C. and 65%±5% relative humidity, uninterrupted, (except for the addition or withdrawal of test samples) for a period of three months or six months. Tablets were removed from the storage conditions at the specified time points and tested for levothyroxine and liothyronine content using high pressure liquid chromatography.

Considering levothyroxine content, shelf life of tablets of Batches S45400121A, S45400121B, S45400221A, S45400221B, S45400321A, and S45400321B was estimated to be at least six months when stored under intermediate testing conditions. Table 25 provides the six-month stability assay results for tablets of Batches S45400121A, S45400121B, S45400221A, S45400221B, S45400321A, and S45400321B stored under intermediate testing conditions. The stability assay showed that greater than 95 percent of the original levothyroxine content was retained in tablets stored under intermediate testing conditions for six months.

Considering liothyronine content, shelf life of tablets of Batches S45400121A, S45400121B, S45400221A, S45400221B, S45400321A, and S45400321B was estimated to be at least six months when stored under intermediate testing conditions. As shown in Table 25, the stability assay showed that greater than 95 percent of the original liothyronine content was retained in tablets stored under intermediate testing conditions for six months.

TABLE 25
Stability assay results under intermediate testing conditions for tablets of Batches S45400121A,
S45400121B, S45400221A, S45400221B, S45400321A, and S45400321B
Thyroid Tablets, USP, 30° C./65% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 130 mg

| | | | | | | |
|---|---|---|---|---|---|---|
| S45400121A | 14 | 0 M | 97.5 | 101.4 | 02:52 | 2% |
| | | 3 M | 99.0 | 102.0 | 04:26 | 1% |
| | | 6 M | 97.3 | 101.6 | 04:49 | 1% |
| S45400121B | 90 | 0 M | 97.5 | 101.4 | 02:52 | 2% |
| | | 3 M | 99.6 | 102.8 | 04:19 | 2% |
| | | 6 M | 96.4 | 100.4 | 05:27 | 2% |
| | | 0 M | 97.9 | 101.1 | 04:10 | 2% |
| S45400221A | 14 | 3 M | 98.4 | 101.1 | 05:31 | 1% |
| | | 6 M | 94.6 | 99.0 | 04:33 | 1% |
| S45400221B | 90 | 0 M | 97.9 | 101.1 | 04:10 | 2% |
| | | 3 M | 99.6 | 102.7 | 04:02 | 2% |
| | | 6 M | 96.3 | 96 | 03:49 | 2% |
| S45400321A | 14 | 0 M | 97.5 | 101.5 | 03:21 | 3% |
| | | 3 M | 98.2 | 101.3 | 03:43 | 1% |
| | | 6 M | 95.6 | 99.6 | 03:20 | 2% |
| S45400321B | 90 | 0 M | 97.5 | 101.5 | 03:21 | 3% |
| | | 3 M | 101.2 | 104.6 | 04:01 | 2% |
| | | 6 M | 96 | 100.9 | 03:50 | 2% |

With reference to the results shown in Table 25, the present inventors surprisingly observed that oral solid dosage forms produced in Batches S45400121A, S45400121B, S45400221A, S45400221B, S45400321A, and S45400321B retained at least about 95% of the original content of levothyroxine when stored under intermediate testing conditions at 30° C.±2° C. and 65%±5% relative humidity for a period of six months.

With reference to the results shown in Table 25, the present inventors also surprisingly observed that oral solid dosage forms produced in Batches S45400121A, S45400121B, S45400221A, S45400221B, S45400321A, and S45400321B retained at least about 97% of the original content of liothyronine when stored under intermediate testing conditions at 30° C.±2° C. and 65%±5% relative humidity for a period of six months.

Accelerated condition stability samples were placed upright in an environmental chamber and maintained in an upright position during the stability testing. As discussed, each tablet was disposed in the sealed container containing either 10 to 20 or 90 to 100 tablets from Batches S45400121A, S45400121B, S45400221A, S45400221B, S45400321A, and S45400321B, and each tablet included about 76 μg of levothyroxine and about 18 μg of liothyronine per tablet. The sealed containers were maintained at 40° C.±2° C. and 75%±5% relative humidity, uninterrupted, (except for the addition or withdrawal of test samples) for a period of 1 month, 2 months, 3 months, 4 months, 5 months or six months. Tablets were removed from the storage conditions at the specified time points and tested for levothyroxine and liothyronine content using high pressure liquid chromatography.

Considering levothyroxine content, shelf life of tablets of Batches S45400121A, S45400121B, S45400221A, S45400221B, S45400321A, and S45400321B was estimated to be at least six months when stored under accelerated testing conditions. Table 26 provides the six-month stability assay results for tablets of Batches S45400121A, S45400121B, S45400221A, S45400221B, S45400321A, and S45400321B stored under accelerated testing conditions. The stability assay showed that greater than 95 percent of the original levothyroxine content was retained in tablets stored under accelerated testing conditions for six months.

Considering liothyronine content, shelf life of tablets of Batches S45400121A, S45400121B, S45400221A, S45400221B, S45400321A, and S45400321B was estimated to be at least six months when stored under accelerated testing conditions. As shown in Table 26, the stability assay showed that greater than 95 percent of the original liothyronine content was retained in tablets stored under accelerated testing conditions for six months.

TABLE 26
Stability assay results under accelerated testing conditions for tablets of Batches S45400121A,
S45400121B, S45400221A, S45400221B, S45400321A, and S45400321B
Thyroid Tablets, USP, 40 C./75% RH

| | Count | Time | Assay (%) | | Disintegration | Moisture |
|---|---|---|---|---|---|---|
| Lot Numbers | (Tablets) | Point | Liothyronine | Levothyroxine | (Min:Sec) | Content |

Strength: 130 mg

| | | | | | | |
|---|---|---|---|---|---|---|
| S45400121A | 14 | 0 M | 97.5 | 101.4 | 02:52 | 2% |
| | | 1 M | 99.2 | 102.8 | 04:42 | 2% |
| | | 2 M | 97.4 | 100.5 | 04:24 | 1% |
| | | 3 M | 99.1 | 102.4 | 03:59 | 1% |
| | | 4 M | 97.8 | 101.5 | 04:50 | 1% |
| | | 5 M | 97.4 | 100.7 | 04:13 | 2% |
| | | 6 M | 95.2 | 98.8 | 04:20 | 2% |
| S45400121B | 90 | 0 M | 97.5 | 101.4 | 02:52 | 2% |
| | | 1 M | 97.5 | 100.7 | 04:41 | 2% |
| | | 2 M | 98.3 | 102.6 | 04:48 | 2% |
| | | 3 M | 99.0 | 102.2 | 06:00 | 2% |
| | | 4 M | 94.7 | 94.4 | 06:14 | 2% |
| | | 5 M | 97.2 | 100.7 | 06:11 | 2% |
| | | 6 M | 94.8 | 97.7 | 05:35 | 2% |
| S45400221A | 14 | 0 M | 97.9 | 101.1 | 04:10 | 2% |
| | | 1 M | 98.5 | 101.8 | 03:41 | 2% |
| | | 2 M | 97.7 | 101.4 | 04:33 | 1% |
| | | 3 M | 99.5 | 102.3 | 04:44 | 1% |
| | | 4 M | 96.9 | 100.3 | 03:37 | 1% |
| | | 5 M | 97.8 | 101.6 | 04:29 | 2% |
| | | 6 M | 95.0 | 99.7 | 04:42 | 2% |
| S45400221B | 90 | 0 M | 97.9 | 101.1 | 04:10 | 2% |
| | | 1 M | 97.6 | 100.7 | 04:54 | 2% |
| | | 2 M | 98.2 | 102.3 | 04:49 | 2% |
| | | 3 M | 99.9 | 103.0 | 05:20 | 2% |
| | | 4 M | 96.0 | 95.8 | 05:03 | 2% |
| | | 5 M | 97.2 | 98.9 | 06:09 | 2% |
| | | 6 M | 98.4 | 98.5 | 04:46 | 2% |
| S45400321A | 14 | 0 M | 97.5 | 101.5 | 03:21 | 3% |
| | | 1 M | 97.9 | 101.1 | 03:26 | 2% |
| | | 2 M | 98.2 | 102.3 | 03:34 | 1% |
| | | 3 M | 98.0 | 100.9 | 03:27 | 1% |
| | | 4 M | 96.4 | 96.8 | 03:58 | 1% |
| | | 5 M | 97.2 | 101.7 | 03:57 | 1% |
| | | 6 M | 95.2 | 99.5 | 03:38 | 2% |
| S45400321B | 90 | 0 M | 97.5 | 101.5 | 03:21 | 3% |
| | | 1 M | 99.3 | 103.4 | 04:24 | 2% |
| | | 2 M | 99.4 | 103.7 | 04:04 | 2% |
| | | 3 M | 99.6 | 102.5 | 05:32 | 2% |
| | | 4 M | 100.9 | 101.4 | 05:05 | 2% |
| | | 5 M | 97.4 | 98.7 | 05:51 | 2% |
| | | 6 M | 96.4 | 96.2 | 06:04 | 2% |

With reference to the results shown in Table 26, the present inventors surprisingly observed that oral solid dosage forms produced in Batches S45400121A, S45400121B, S45400221A, S45400221B, S45400321A, and S45400321B retained at least about 95% of the original content of levothyroxine when stored under accelerated testing conditions at 40° C.±2° C. and 75%±5% relative humidity for a period of six months.

With reference to the results shown in Table 26, the present inventors also surprisingly observed that oral solid dosage forms produced in Batches S45400121A, S45400121B, S45400221A, S45400221B, S45400321A, and S45400321B retained at least 97% of the original content of liothyronine when stored under accelerated testing conditions at 40° C.±2° C. and 75%±5% relative humidity for a period of six months.

The following numbered clauses are directed to various non-limiting embodiments of inventions according to the present disclosure:
1. A ready to use pharmaceutical solid dosage form comprising, by weight:
45% to 55% porcine thyroid powder;
35% to 49% microcrystalline cellulose; and
at least one excipient selected from a lubricant, a glidant, a diluent, and a disintegrant.
2. The ready to use pharmaceutical solid dosage form of Clause 1, wherein the porcine thyroid powder comprises 7 μg to 230 μg of levothyroxine.
3. The ready to use pharmaceutical solid dosage form of Clause 1 or Clause 2, wherein the porcine thyroid powder comprises 1.6 μg to 55 μg of liothyronine.
4. The ready to use pharmaceutical solid dosage form of Clause 1, wherein the porcine thyroid powder comprises about 9.5 μg of levothyroxine and about 2.2 g of liothyronine.
5. The ready to use pharmaceutical solid dosage form of Clause 1, wherein the porcine thyroid powder comprises about 19 μg of levothyroxine and about 4.5 μg of liothyronine.
6. The ready to use pharmaceutical solid dosage form of Clause 1, wherein the porcine thyroid powder comprises about 38 μg of levothyroxine and about 9 μg of liothyronine.
7. The ready to use pharmaceutical solid dosage form of Clause 1, wherein the porcine thyroid powder comprises about 57 μg of levothyroxine and about 13.5 μg of liothyronine.
8. The ready to use pharmaceutical solid dosage form of Clause 1, wherein the porcine thyroid powder comprises about 76 μg of levothyroxine and about 18 μg of liothyronine.
9. The ready to use pharmaceutical solid dosage form of Clause 1, wherein the porcine thyroid powder comprises about 114 μg of levothyroxine and about 27 μg of liothyronine.
10. The ready to use pharmaceutical solid dosage form of Clause 1, wherein the porcine thyroid powder comprises about 152 μg of levothyroxine and about 36 μg of liothyronine.
11. The ready to use pharmaceutical solid dosage form of Clause 1, wherein the porcine thyroid powder comprises about 190 μg of levothyroxine and about 45 μg of liothyronine.
12. The ready to use pharmaceutical solid dosage form of any of Clauses 1-11, wherein the pharmaceutical solid dosage form comprises mannitol.
13. The ready to use pharmaceutical solid dosage form of any of Clauses 1-12, wherein the pharmaceutical solid dosage form comprises at least one lubricant selected from calcium stearate and magnesium stearate.
14. The ready to use pharmaceutical solid dosage form of any of Clauses 1-13, wherein the pharmaceutical solid dosage form comprises, by weight, 2% to 3% colloidal silicon dioxide.
15. The ready to use pharmaceutical solid dosage form of any of Clauses 1-14, wherein the pharmaceutical solid dosage form comprises, by weight, 2% to 3% sodium starch glycolate.
16. The ready to use pharmaceutical solid dosage form of any of Clauses 1-15, wherein the pharmaceutical solid dosage form comprises, by weight, 48% to 52% porcine thyroid powder.
17. The ready to use pharmaceutical solid dosage form of any of Clauses 1-16, wherein the pharmaceutical solid dosage form comprises, by weight, 42% to 46% microcrystalline cellulose.
18. The ready to use pharmaceutical solid dosage form of any of Clauses 1-17, provided that a levothyroxine content in the pharmaceutical solid dosage form is at least 90%, at least 92%, or at least 95% of an initial levothyroxine content in the pharmaceutical solid dosage form after the solid dosage form has been stored for 9 months at a temperature of about 23° C. to about 27° C. and a relative humidity ranging from about 55% to about 65%.
19. The ready to use pharmaceutical solid dosage form of any of Clauses 1-17, provided that a levothyroxine content in the pharmaceutical solid dosage form is at least 90%, at least 92%, or at least 95% of an initial levothyroxine content in the pharmaceutical solid dosage form after the solid dosage form has been stored for 12 months at a temperature of about 23° C. to about 27° C. and a relative humidity ranging from about 55% to about 65%,
20. The ready to use pharmaceutical solid dosage form of any of Clauses 1-17, provided that a levothyroxine content in the pharmaceutical solid dosage form is at least 90%, at least 92%, or at least 95% of an initial levothyroxine content in the pharmaceutical solid dosage form after the solid dosage form has been stored for 18 months at a temperature of about 23° C. to about 27° C. and a relative humidity ranging from about 55% to about 65%
21. The ready to use pharmaceutical solid dosage form of any of Clauses 1-17, provided that a levothyroxine content in the pharmaceutical solid dosage form is at least 90%, at least 92%, or at least 95% of an initial levothyroxine content in the pharmaceutical solid dosage form after the solid dosage form has been stored for 24 months at a temperature of about 23° C. to about 27° C. and a relative humidity ranging from about 55% to about 65%
22. The ready to use pharmaceutical solid dosage form of any of Clauses 1-17, provided that a levothyroxine content in the pharmaceutical solid dosage form is at least 90%, at least 92%, or at least 95% of an initial levothyroxine content in the pharmaceutical solid dosage form after the solid dosage form has been stored for 3 months at a temperature of about 28° C. to about 32° C. and a relative humidity ranging from about 60% to about 70%.
23. The ready to use pharmaceutical solid dosage form of any of Clauses 1-17, provided that a levothyroxine content in the pharmaceutical solid dosage form is at least 90%, at least 92%, or at least 95% of an initial levothyroxine content in the pharmaceutical solid dosage form after the solid dosage form has been stored for 6 months at a temperature of about 28° C. to about 32° C. and a relative humidity ranging from about 60% to about 70%.
24. The ready to use pharmaceutical solid dosage form of any of Clauses 1-17, provided that a levothyroxine content in the pharmaceutical solid dosage form is at least 90%, at least 92%, or at least 95% of an initial levothyroxine content in the pharmaceutical solid dosage form after the pharmaceutical solid dosage form has been stored for 3 months at a temperature ranging from about 38° C. to about 42° C. and a relative humidity ranging from about 70% to about 80%.
25. The ready to use pharmaceutical solid dosage form of any of Clauses 1-17, provided that a levothyroxine content in the pharmaceutical solid dosage form is at least 90%, at least 92%, or at least 95% of an initial levothyroxine content in the pharmaceutical solid dosage form after the pharmaceutical solid dosage form has been stored for 6 months at a temperature ranging from about 38° C. to about 42° C. and a relative humidity ranging from about 70% to about 80%.
26. The ready to use pharmaceutical solid dosage form of any of Clauses 1-25, provided that a liothyronine content in the pharmaceutical solid dosage form is at least 90%, at least 92%, or at least 95% of an initial liothyronine content in the pharmaceutical solid dosage form after the solid dosage form has been stored for 9 months at a temperature of about 23° C. to about 27° C. and a relative humidity ranging from about 55% to about 65%.
27. The ready to use pharmaceutical solid dosage form of any of Clauses 1-25, provided that the liothyronine content in the pharmaceutical solid dosage form is at least 90%, at least 92, or at least 95% of an initial liothyronine content in the pharmaceutical solid dosage form after the solid dosage form has been stored for 12 months at a temperature of about 23° C. to about 27° C. and a relative humidity ranging from about 55% to about 65%.
28. The ready to use pharmaceutical solid dosage form of any of Clauses 1-25, provided that the liothyronine content in the pharmaceutical solid dosage form is at least 90%, at least 92, or at least 95% of an initial liothyronine content in the pharmaceutical solid dosage form after the solid dosage form has been stored for 18 months at a temperature of about 23° C. to about 27° C. and a relative humidity ranging from about 55% to about 65%.
29. The ready to use pharmaceutical solid dosage form of any of Clauses 1-25, provided that the liothyronine content in the pharmaceutical solid dosage form is at least 90%, at least 92, or at least 95% of an initial liothyronine content in the pharmaceutical solid dosage form after the solid dosage form has been stored for 24 months at a temperature of about 23° C. to about 27° C. and a relative humidity ranging from about 55% to about 65%.
30. The ready to use pharmaceutical solid dosage form of any of Clauses 1-25, provided that a liothyronine content in the pharmaceutical solid dosage form is at least 90%, at least 92%, or at least 95% of an initial liothyronine content in the pharmaceutical solid dosage form after the solid dosage form has been stored for 3 months at a temperature of about 28° C. to about 32° C. and a relative humidity ranging from about 60% to about 70%.
31. The ready to use pharmaceutical solid dosage form of any of Clauses 1-25, provided that a liothyronine content in the pharmaceutical solid dosage form is at least 90%, at least 92%, or at least 95% of an initial liothyronine content in the pharmaceutical solid dosage form after the solid dosage form has been stored for 6 months at a temperature of about 28° C. to about 32° C. and a relative humidity ranging from about 60% to about 70%.
32. The ready to use pharmaceutical solid dosage form of any of Clauses 1-25, provided that a liothyronine content in the pharmaceutical solid dosage form is at least 90%, at least 92%, or at least 95% of an initial liothyronine content in the pharmaceutical solid dosage form after the pharmaceutical solid dosage form has been stored for 3 months at a temperature ranging from about 38° C. to about 42° C. and a relative humidity ranging from about 70% to about 80%.
33. The ready to use pharmaceutical solid dosage form of any of Clauses 1-25, provided that the liothyronine content in the pharmaceutical solid dosage form is at least 90%, at least 92%, or at least 95% of an initial liothyronine content in the pharmaceutical solid dosage form after the pharmaceutical solid dosage form has been stored for 6 months at a temperature ranging from about 38° C. to about 42° C. and a relative humidity ranging from about 70% to about 80%
34. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial levothyroxine content in the oral solid dosage form after the oral solid dosage form has been stored for 9 months at a temperature ranging from about 23° C. to about 27° C. and a relative humidity ranging from about 55% to about 65%.
35. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial levothyroxine content in the oral solid dosage form after the oral solid dosage form has been stored for 12 months at a temperature ranging from about 23° C. to about 27° C. and a relative humidity ranging from about 55% to about 65%.
36. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial levothyroxine content in the oral solid dosage form after the oral solid dosage form has been stored for 18 months at a temperature ranging from about 23° C. to about 27° C. and a relative humidity ranging from about 55% to about 65%.
37. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial levothyroxine content in the oral solid dosage form after the oral solid dosage form has been stored for 24 months at a temperature ranging from about 23° C. to about 27° C. and a relative humidity ranging from about 55% to about 65%.
38. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial levothyroxine content in the oral solid dosage form after the oral solid dosage form has been stored for 3 months at a temperature ranging from about 28° C. to about 32° C. and a relative humidity ranging from about 60% to about 70%.
39. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial levothyroxine content in the oral solid dosage form after the oral solid dosage form has been stored for 6 months at a temperature ranging from about 28° C. to about 32° C. and a relative humidity ranging from about 60% to about 70%.
40. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial levothyroxine content in the oral solid dosage form after the oral solid dosage form has been stored for 3 months at a temperature ranging from 38° C. to about 42° C. and a relative humidity ranging from about 70% to about 80%.
41. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial levothyroxine content in the oral solid dosage form after the oral solid dosage form has been stored for 6 months at a temperature ranging from 38° C. to about 42° C. and a relative humidity ranging from about 70% to about 80%.
42. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial liothyronine content in the oral solid dosage form after the oral solid dosage form has been stored for 9 months at a temperature ranging from about 23° C. to about 27° C. and a relative humidity ranging from about 55% to about 65%.
43. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial liothyronine content in the oral solid dosage form after the oral solid dosage form has been stored for 12 months at a temperature ranging from about 23° C. to about 27° C. and a relative humidity ranging from about 55% to about 65%.
44. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial liothyronine content in the oral solid dosage form after the oral solid dosage form has been stored for 18 months at a temperature ranging from about 23° C. to about 27° C. and a relative humidity ranging from about 55% to about 65%.
45. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial liothyronine content in the oral solid dosage form after the oral solid dosage form has been stored for 24 months at a temperature ranging from about 23° C. to about 27° C. and a relative humidity ranging from about 55% to about 65%.
46. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial liothyronine content in the oral solid dosage form after the oral solid dosage form has been stored for 3 months at a temperature ranging from about 28° C. to about 32° C. and a relative humidity ranging from about 60% to about 70%.
47. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial liothyronine content in the oral solid dosage form after the oral solid dosage form has been stored for 6 months at a temperature ranging from about 28° C. to about 32° C. and a relative humidity ranging from about 60% to about 70%.
48. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial liothyronine content in the oral solid dosage form after the oral solid dosage form has been stored for 3 months at a temperature ranging from 38° C. to about 42° C. and a relative humidity ranging from about 70% to about 80%.
49. The ready to use pharmaceutical solid dosage form of any of Clauses 1-25, provided that when a plurality of the oral solid dosage forms and at least about 2 grams of desiccant are placed in a container sealed with a closure, each oral solid dosage form retains at least 90%, at least 92%, or at least 95% of an initial liothyronine content in the oral solid dosage form after the oral solid dosage form has been stored for 6 months at a temperature ranging from 38° C. to about 42° C. and a relative humidity ranging from about 70% to about 80%.
50. The ready to use pharmaceutical solid dosage form of any of Clauses 34-49, wherein the container comprises an internal volume of about 15 cm³ to about 45 cm³.
51. The ready to use pharmaceutical solid dosage form of Clause 50, wherein about 10 to about 20 of the oral solid dosage forms are disposed in the container.
52. The ready to use pharmaceutical solid dosage form of any of Clauses 34-49, wherein the container comprises an internal volume of about 60 cm³ to about 90 cm³.
53. The ready to use pharmaceutical solid dosage form of Clause 52, wherein about 90 to about 100 of the oral solid dosage forms are disposed in the container.
54. A packaged immediate release oral solid dosage form product comprising:
an immediate release oral solid dosage form including, by weight,
   45% to 55% porcine thyroid powder;
   35% to 49% microcrystalline cellulose; and
   at least one excipient selected from a lubricant, a glidant, a diluent, and a disintegrant;
a container closure system comprising a container and a sealing closure;
provided that a plurality of the oral solid dosage forms and at least about 2 grams of desiccant are enclosed in the container and sealed with the sealing closure; and
provided that each oral solid dosage form enclosed in the container retains at least 90%, at least 92%, or at least 95% of an initial levothyroxine content in the oral solid dosage form after the oral solid dosage form has been stored for 9 months at a temperature ranging from about 23° C. to about 27° C. and a relative humidity ranging from about 55% to about 65%.
55. The packaged immediate release oral solid dosage form product of Clause 54, wherein the porcine thyroid powder comprises 7 μg to 230 μg of levothyroxine.
56. The packaged immediate release oral solid dosage form product of Clause 54, wherein the porcine thyroid powder comprises 1.6 μg to 55 μg of liothyronine.
57. The packaged immediate release oral solid dosage form product of Clause 54, wherein the porcine thyroid powder comprises about 9.5 μg of levothyroxine and about 2.2 μg of liothyronine.
58. The packaged immediate release oral solid dosage form product of Clause 54, wherein the porcine thyroid powder comprises about 19 μg of levothyroxine and about 4.5 μg of liothyronine.
59. The packaged immediate release oral solid dosage form product of Clause 54, wherein the porcine thyroid powder comprises about 38 μg of levothyroxine and about 9 μg of liothyronine.
60. The packaged immediate release oral solid dosage form product of Clause 54, wherein the porcine thyroid powder comprises about 57 μg of levothyroxine and about 13.5 μg of liothyronine.
61. The packaged immediate release oral solid dosage form product of Clause 54, wherein the porcine thyroid powder comprises about 76 μg of levothyroxine and about 18 μg of liothyronine.
62. The packaged immediate release oral solid dosage form product of Clause 54, wherein the porcine thyroid powder comprises about 114 μg of levothyroxine and about 27 μg of liothyronine.
63. The packaged immediate release oral solid dosage form product of Clause 54, wherein the porcine thyroid powder comprises about 152 μg of levothyroxine and about 36 μg of liothyronine.
64. The packaged immediate release oral solid dosage form product of Clause 54, wherein the porcine thyroid powder comprises about 190 μg of levothyroxine and about 45 μg of liothyronine.
65. The packaged immediate release oral solid dosage form product of any of Clauses 54-64, wherein the oral solid dosage form comprises mannitol.
66. The packaged immediate release oral solid dosage form product of any of Clauses 54-65, wherein the oral solid dosage form comprises at least one lubricant selected from calcium stearate and magnesium stearate.
67. The packaged immediate release oral solid dosage form product of any of Clauses 54-66, wherein the oral solid dosage form comprises, by weight, 2% to 3% colloidal silicon dioxide.
68. The packaged immediate release oral solid dosage form product of any of Clauses 54-67, wherein the oral solid dosage form comprises, by weight, 2% to 3% sodium starch glycolate.
69. The packaged immediate release oral solid dosage form product of any of Clauses 54-68, wherein the oral solid dosage form comprises, by weight, 48% to 52% porcine thyroid powder.
70. The packaged immediate release oral solid dosage form product of any of Clauses 54-69, wherein the oral solid dosage form comprises, by weight, 42% to 46% microcrystalline cellulose.
71. The packaged immediate release oral solid dosage form product of any of Clauses 54-70, wherein the container comprises an internal volume of about 15 cm³ to about 45 cm³.
72. The packaged immediate release oral solid dosage form product of Clause 71, wherein about 10 to about 20 of the oral solid dosage forms are disposed in the container.
73. The packaged immediate release oral solid dosage form product of any of Clauses 54-70, wherein the container comprises an internal volume of about 60 cm³ to about 90 cm³.
74. The packaged immediate release oral solid dosage form product of Clause 73, wherein about 90 to about 100 of the oral solid dosage forms are disposed in the container.
75. The packaged immediate release oral solid dosage form product of any of Clauses 54-74, provided that each oral solid dosage form enclosed in the container retains at least 90%, at least 92%, or at least 95% of an initial levothyroxine content in the oral solid dosage form after the oral solid dosage form has been stored for 12 months at a temperature ranging from about 23° C. to about 27° C. and a relative humidity ranging from about 55% to about 65%.
76. The packaged immediate release oral solid dosage form product of any of Clauses 54-74, provided that each oral solid dosage form enclosed in the container retains at least 90%, at least 92%, or at least 95% of an initial levothyroxine content in the oral solid dosage form after the oral solid dosage form has been stored for 18 months at a temperature ranging from about 23° C. to about 27° C. and a relative humidity ranging from about 55% to about 65%.
77. The packaged immediate release oral solid dosage form product of any of Clauses 54-74, provided that each oral solid dosage form enclosed in the container retains at least 90%, at least 92%, or at least 95% of an initial levothyroxine content in the oral solid dosage form after the oral solid dosage form has been stored for 24 months at a temperature ranging from about 23° C. to about 27° C. and a relative humidity ranging from about 55% to about 65%.
78. The packaged immediate release oral solid dosage form product of any of Clauses 54-74, provided that each oral solid dosage form enclosed in the container retains at least 90%, at least 92%, or at least 95% of an initial levothyroxine content in the oral solid dosage form after the oral solid dosage form has been stored for 3 months at a temperature ranging from about 28° C. to about 32° C. and a relative humidity ranging from about 60% to about 70%.
79. The packaged immediate release oral solid dosage form product of any of Clauses 54-74, provided that each oral solid dosage form enclosed in the container retains at least 90%, at least 92%, or at least 95% of an initial levothyroxine content in the oral solid dosage form after the oral solid dosage form has been stored for 6 months at a temperature ranging from about 28° C. to about 32° C. and a relative humidity ranging from about 60% to about 70%
80. The packaged immediate release oral solid dosage form product of any of Clauses 54-74, provided that each oral solid dosage form enclosed in the container retains at least 90%, at least 92%, or at least 95% of an initial levothyroxine content in the oral solid dosage form after the oral solid dosage form has been stored for 3 months at a temperature ranging from about 38° C. to about 42° C. and a relative humidity ranging from about 70% to about 80%.
81. The packaged immediate release oral solid dosage form product of any of Clauses 54-74, provided that each oral solid dosage form enclosed in the container retains at least 90%, at least 92%, or at least 95% of an initial levothyroxine content in the oral solid dosage form after the oral solid dosage form has been stored for 6 months at a temperature ranging from about 38° C. to about 42° C. and a relative humidity ranging from about 70% to about 80%.
82. The packaged immediate release oral solid dosage form product of any of Clauses 54-81, provided that each oral solid dosage form enclosed in the container retains at least 90%, at least 92%, or at least 95% of an initial liothyronine content in the oral solid dosage form after the oral solid dosage form has been stored for 9 months at a temperature ranging from about 23° C. to about 27° C. and a relative humidity ranging from about 55% to about 65%.
83. The packaged immediate release oral solid dosage form product of any of Clauses 54-81, provided that each oral solid dosage form enclosed in the container retains at least 90%, at least 92%, or at least 95% of an initial liothyronine content in the oral solid dosage form after the oral solid dosage form has been stored for 12 months at a temperature ranging from about 23° C. to about 27° C. and a relative humidity ranging from about 55% to about 65%.
84. The packaged immediate release oral solid dosage form product of any of Clauses 54-81, provided that each oral solid dosage form enclosed in the container retains at least 90%, at least 92%, or at least 95% of an initial liothyronine content in the oral solid dosage form after the oral solid dosage form has been stored for 18 months at a temperature ranging from about 23° C. to about 27° C. and a relative humidity ranging from about 55% to about 65%.
85. The packaged immediate release oral solid dosage form product of any of Clauses 54-81, provided that each oral solid dosage form enclosed in the container retains at least 90%, at least 92%, or at least 95% of an initial liothyronine content in the oral solid dosage form after the oral solid dosage form has been stored for 24 months at a temperature ranging from about 23° C. to about 27° C. and a relative humidity ranging from about 55% to about 65%.
86. The packaged immediate release oral solid dosage form product of any of Clauses 54-81, provided that each oral solid dosage form enclosed in the container retains at least 90%, at least 92%, or at least 95% of an initial liothyronine content in the oral solid dosage form after the oral solid dosage form has been stored for 3 months at a temperature ranging from about 28° C. to about 32° C. and a relative humidity ranging from about 60% to about 70%.
87. The packaged immediate release oral solid dosage form product of any of Clauses 54-81, provided that each oral solid dosage form enclosed in the container retains at least 90%, at least 92%, or at least 95% of an initial liothyronine content in the oral solid dosage form after the oral solid dosage form has been stored for 6 months at a temperature ranging from about 28° C. to about 32° C. and a relative humidity ranging from about 60% to about 70%.
88. The packaged immediate release oral solid dosage form product of any of Clauses 54-81, provided that each oral solid dosage form enclosed in the container retains at least 90%, at least 92%, or at least 95% of an initial liothyronine content in the oral solid dosage form after the oral solid dosage form has been stored for 3 months at a temperature ranging from about 38° C. to about 42° C. and a relative humidity ranging from about 70% to about 80%.
89. The packaged immediate release oral solid dosage form product of any of Clauses 54-81, provided that each oral solid dosage form enclosed in the container retains at least 90%, at least 92%, or at least 95% of an initial liothyronine content in the oral solid dosage form after the oral solid dosage form has been stored for 6 months at a temperature ranging from about 38° C. to about 42° C. and a relative humidity ranging from about 70% to about 80%.
90. A method of treatment to address hypothyroidism, the method comprising administering an immediate release oral solid dosage form to a subject in need thereof, wherein the immediate release oral solid dosage form comprises, in weight percentages:
   45% to 55% porcine thyroid powder;
   35% to 49% microcrystalline cellulose; and
   at least one excipient selected from a lubricant, a glidant, a diluent, and a disintegrant.
91. A method of treatment to address hypothyroidism, the method comprising administering to a subject in need thereof the ready to use pharmaceutical solid dosage form of any of Clauses 1-53.

1.-79. (canceled)

80. A ready to use pharmaceutical solid dosage form comprising, by weight:
45% to 55% porcine thyroid powder;
35% to 49% microcrystalline cellulose; and
at least one excipient selected from a lubricant, a glidant, a diluent, and a disintegrant.

81. The ready to use pharmaceutical solid dosage form of claim 80, wherein the porcine thyroid powder comprises 7 μg to 230 μg of levothyroxine.

82. The ready to use pharmaceutical solid dosage form of claim 80, wherein the porcine thyroid powder comprises 1.6 μg to 55 μg of liothyronine.

83. The ready to use pharmaceutical solid dosage form of claim 80, wherein the pharmaceutical solid dosage form comprises mannitol.

84. The ready to use pharmaceutical solid dosage form of claim 80, wherein the pharmaceutical solid dosage form comprises at least one lubricant selected from calcium stearate and magnesium stearate.

85. The ready to use pharmaceutical solid dosage form of claim 80, wherein the pharmaceutical solid dosage form comprises, by weight, 2% to 3% colloidal silicon dioxide.

86. The ready to use pharmaceutical solid dosage form of claim 80, wherein the pharmaceutical solid dosage form comprises, by weight, 2% to 3% sodium starch glycolate.

87. The ready to use pharmaceutical solid dosage form of claim 80, provided that a levothyroxine content in the pharmaceutical solid dosage form is at least 90% of an initial levothyroxine content in the pharmaceutical solid dosage form after the solid dosage form has been stored for 9 months at a temperature of about 23° C. to about 27° C. and a relative humidity ranging from about 55% to about 65%.

88. The ready to use pharmaceutical solid dosage form of claim 80, provided that a levothyroxine content in the pharmaceutical solid dosage form is at least 90% of an initial levothyroxine content in the pharmaceutical solid dosage form after the solid dosage form has been stored for 3 months at a temperature of about 28° C. to about 32° C. and a relative humidity ranging from about 60% to about 70%.

89. The ready to use pharmaceutical solid dosage form of claim 80, provided that a levothyroxine content in the pharmaceutical solid dosage form is at least 90% of an initial levothyroxine content in the pharmaceutical solid dosage form after the pharmaceutical solid dosage form has been stored for 3 months at a temperature ranging from about 38° C. to about 42° C. and a relative humidity ranging from about 70% to about 80%.

90. The ready to use pharmaceutical solid dosage form of claim 80, provided that a liothyronine content in the pharmaceutical solid dosage form is at least 90% of an initial liothyronine content in the pharmaceutical solid dosage form after the solid dosage form has been stored for 9 months at a temperature of about 23° C. to about 27° C. and a relative humidity ranging from about 55% to about 65%.

91. The ready to use pharmaceutical solid dosage form of claim 80, provided that a liothyronine content in the pharmaceutical solid dosage form is at least 90% of an initial liothyronine content in the pharmaceutical solid dosage form after the solid dosage form has been stored for 12 months at a temperature of about 23° C. to about 27° C. and a relative humidity ranging from about 55% to about 65%.

92. The ready to use pharmaceutical solid dosage form of claim 80, provided that a liothyronine content in the pharmaceutical solid dosage form is at least 90% of an initial liothyronine content in the pharmaceutical solid dosage form after the solid dosage form has been stored for 3 months at a temperature of about 28° C. to about 32° C. and a relative humidity ranging from about 60% to about 70%.

93. The ready to use pharmaceutical solid dosage form of claim 80, provided that a liothyronine content in the pharmaceutical solid dosage form is at least 90% of an initial liothyronine content in the pharmaceutical solid dosage form after the pharmaceutical solid dosage form has been stored for 3 months at a temperature ranging from about 38° C. to about 42° C. and a relative humidity ranging from about 70% to about 80%.
